(19) United States
(12) Patent Application Publication
(10) Pub. No.: US 2026/0348962 A1
(43) Pub. Date: Nov. 5, 2026

(54) NONINVASIVE TEST FOR EARLY DETECTION OF PANCREATIC DUCTAL ADENOCARCINOMA

(71) Applicant: Cedars-Sinai Medical Center, Los Angeles, CA (US)

(72) Inventors: Nirdesh K. Gupta, Valencia, CA (US); Jian Tajbakhsh, Woodland Hills, CA (US)

(73) Assignee: Cedars-Sinai Medical Center, Los Angeles, CA (US)

(21) Appl. No.: 19/699,932

(22) Filed: Jun. 5, 2026

Related U.S. Application Data
(63) Continuation of application No. PCT/US2025/044228, filed on Aug. 29, 2025.
(60) Provisional application No. 63/689,166, filed on Aug. 30, 2024.

(51) Int. Cl. G01N 33/57525; G01N 21/76; G01N 33/53; G01N 33/575; G01N 33/68

(52) U.S. Cl. CPC G01N 33/57525; G01N 21/76; G01N 33/5306; G01N 33/57585; G01N 33/5759; G01N 33/6893; G01N 2333/705; G01N 2400/00

(57) ABSTRACT

Described herein are methods of calculating a pancreatic cancer risk score and methods of stratifying patients; for example, into two groups-patients who can be further monitored by also repeating the test in intervals of several months, and patients who need further testing by confirmatory methods. Also described are methods of further confirming pancreatic cancer, and methods of treating pancreatic cancer.

### CROSS-REFERENCE TO RELATED APPLICATIONS

This application includes a claim of priority under 35 U.S.C. § 119 (e) to U.S. provisional patent application No. 63/689,166, filed Aug. 30, 2024, the entirety of which is hereby incorporated by reference.

### REFERENCE TO SEQUENCE LISTING

This application contains a Sequence Listing submitted as a computer readable form named “065472_000986WOPT.xml”, having a size in bytes of 3,170 bytes, and created on Aug. 26, 2025 (production date). The information contained in this computer readable form is hereby incorporated by reference in its entirety.

### FIELD OF INVENTION

This invention relates to detection and treatment of pancreatic cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Despite considerable progress in understanding pancreatic cancer, especially its most common type, pancreatic ductal adenocarcinoma (PDAC), the disease remains lethal with a median patients' survival of only 5-6 months, and exceptionally low 5-year survival of around 9%. Multitude reasons for such a bleak prognosis exist—from late presentation of the disease due to lack of specific clinical symptoms in the early stages, to the currently inefficient therapeutic approaches. It is likely that both would be improved if PDAC could be detected earlier, in pre-symptomatic stage. Currently, the only PDAC biomarker in widespread clinical use, serum CA19.9, suffers from false-negative results in patients with Lewis-negative genotype (5-10% of the Caucasian population). In addition, it is elevated in various benign and malignant pancreatic and hepatobiliary diseases, unrelated cystic and inflammatory diseases, and its overall low positive predictive value impedes its application as a biomarker for early detection of PDAC.

Accordingly, there is a need in the art for early and noninvasive detection of PDAC.

### SUMMARY OF THE INVENTION

The following embodiments and aspects thereof are described and illustrated in conjunction with compositions and methods which are meant to be exemplary and illustrative, not limiting in scope.

Various embodiments provide for a method of calculating a risk score for a subject, comprising: obtaining urine concentrations of lymphatic vessel endothelial hyaluronan receptor 1 (LYVE1) protein, and regenerating family member 1 beta (REG1B) protein; obtaining the subject's age; obtaining blood concentration of CA19.9 carbohydrate; and calculating the risk score using the following Formula I:

$$\frac{1}{( 1 + {e}^{( - ( β0 ) + ( β 1 * L ) + ( β 2 * R ) + ( β 3 * C ) + ( β 4 * A ) ) )} )} , \qquad [ Formula I ]$$

wherein
   L is log_{c}(concentration of LYVE1+0.01),
   R is log_{c}(concentration REG1B+0.01),
   C is log_{c}(concentration CA19.9-+0.01), and
   A is age of subject, and
wherein the calculation for β is: β=b*SD_{IV}*R/SD_{LogitY} and wherein
   β is the standardized weight,
   b is the unstandardized weight,
   SD_{IV} is the standard deviation of the independent variable,
   R is the Pearson correlation coefficient, and
   SD_{LogitY} is the standard deviation of the log odds predicted probability, and wherein β0 is the constant, β1 is the standardized weight for LYVE1, β2 is the standardized weight for REG1B, β3 is the standardized weight for CA19.9 and β4 is the standardized weight for age.

Various embodiments provide for a method of calculating a risk score for a subject, comprising: obtaining urine concentrations of lymphatic vessel endothelial hyaluronan receptor 1 (LYVE1) protein, and regenerating family member 1 beta (REG1B) protein; obtaining the subject's age; obtaining blood concentration of CA19.9 carbohydrate; and calculating the risk score using the following Formula Ia:

$$\frac{1}{( 1 + {e}^{( - ( - 6.895079 - ( 0.007487015 * L ) + ( 0.3566643 * R ) + ( 0.7100223 * C ) + ( 0.0399479 * A ) ) )} )}$$

wherein
L is log_{c}(concentration of LYVE1+0.01),
R is log_{c}(concentration REG1B+0.01),
C is log_{c}(concentration CA19.9 protein+0.01), and
A is age of subject.

In various embodiments, the method can further comprise comparing the risk score to a threshold score; and stratifying a subject as having pancreatic cancer if the risk score is equal to or higher than the threshold score, or stratifying a subject as not having pancreatic cancer if the risk score is lower than the threshold score. In various embodiments, the threshold score can be 0.412.

In various embodiments, the method does not include obtaining urine concentrations of TFF1.

Various embodiments provide for a method of testing a subject in need thereof, comprising: detecting concentration of LYVE1 protein in a urine sample and comparing the LYVE1 protein concentration to a reference range for a healthy subject, or comparing the LYVE1 protein concentration to a biological range for a cancer subject; detecting concentration of REG1B protein in a urine sample and comparing the REG1B protein concentration to a reference range for a healthy subject, or comparing the REG1B protein concentration to a biological range for a cancer subject; detecting concentration of CA19.9 carbohydrate in a blood sample; and obtaining the subject's age.

In various embodiments, a reference range for LYVE1 in a healthy subject can be 1.56 ng/ml-79.63 ng/ml. In various embodiments, a reference range for REG1B in a healthy subject can be 7.81 ng/ml-97.93 ng/ml.

In various embodiments, a biological range for LYVE1 in a cancer subject can be 3.59-100.00 ng/ml. In various embodiments, a biological range for REG1B in a cancer subject can be 7.81-500.00 ng/ml.

In various embodiments, a reportable range for LYVE1 can be 1.56 ng/ml-100.00 ng/ml. In various embodiments, a reportable range for REG1B can be 7.81 ng/ml-500 ng/ml.

In various embodiments, obtaining urine concentrations of LYVE1 and REG1B can comprise: contacting a urine sample, obtained from the subject, to an assay, the assay comprising: one enzyme-linked immunosorbent assay (ELISA) for measuring the concentrations of LYVE1 and REG1B, or two ELISAs, a first ELISA for measuring the concentration of LYVE1 and a second ELISA for measuring the concentration of REG1B.

In various embodiments, the one ELISA can comprise an antibody that specifically binds to LYVE1 and an antibody that specifically binds to REG1B, or the two ELISAs can comprise the first ELISA comprising an antibody that specifically binds to LYVE1 and the second ELISA comprising an antibody that specifically binds to REG1B.

In various embodiments, the one ELISA can comprise a primary capture antibody that specifically binds to LYVE1 and a primary capture antibody that specifically binds to REG1B, or the two ELISAs can comprise the first ELISA comprising a primary capture antibody that specifically binds to LYVE1 and the second ELISA comprising a primary capture antibody that specifically binds to REG1B.

In various embodiments, the one ELISA further can comprise a secondary detection antibody pair that specifically binds to LYVE1 including an additional antibody conjugated to a reporter molecule and a secondary detection antibody pre-conjugated with a reporter molecule that specifically binds to REG1B, or the two ELISAs can comprise the first ELISA further comprising a secondary detection antibody pair that specifically binds to LYVE1 including an additional antibody conjugated to a reporter molecule and the second ELISA further comprising a secondary detection antibody pre-conjugated with a reporter molecule that specifically binds to REG1B.

In various embodiments, obtaining urine concentrations of LYVE1 and REG1B can comprise using an antibody that specifically binds to LYVE1 and an antibody that specifically binds to REG1B.

In various embodiments, the urine sample or blood sample can be obtained from a human subject having or suspected of having pancreatic ductal adenocarcinoma (PDAC), or a human subject having one or more genetic predispositions to PDAC development, a human subject having one or more symptoms of pancreatic cancer.

In various embodiments, the pancreatic ductal adenocarcinoma can be stage I, stage II, stage III or stage IV pancreatic ductal adenocarcinoma.

In various embodiments, obtaining blood concentration of CA19.9 carbohydrate can comprise contacting a blood sample, obtained from the subject, to an enzyme-linked immunosorbent assay (ELISA) or a chemiluminescence assay for measuring the concentration of CA19.9 carbohydrate. In various embodiments, obtaining blood concentration of CA19.9 protein can comprise using an immunoassay.

In various embodiments, the method does not include obtaining urine concentrations of TFF1.

In various embodiments, the method can comprise stratifying a subject as having pancreatic cancer if the risk score is equal to or higher than the threshold score and performing one or more imaging test or one or more biopsies on the subject to further confirm the presence of PDAC.

In various embodiments, the method can comprise stratifying a subject as having pancreatic cancer if the risk score is equal to or higher than the threshold score and further comprising administering PDAC therapy to the subject.

Various embodiments provide for a kit for testing for pancreatic ductal adenocarcinoma comprising: one enzyme-linked immunosorbent assay (ELISA) for measuring the concentration of Lymphatic vessel endothelial hyaluronan receptor 1 (LYVE1) and Regenerating islet-derived 1B (REG1B), or two ELISAs, a first ELISA for measuring concentration of LYVE1, and a second ELISA for measuring concentration of REG1B.

In various embodiments, the kit can further comprise one or more solvents for diluting LYVE1 and REG1B proteins or protein concentrations in a urine sample.

In various embodiments, antibodies specific to LYVEland REG1B can be immobilized on a solid surface. In various embodiments, antibodies specific to LYVE1 and REG1B can be immobilized on a microtiter plate. In various embodiments, the ELISA can be a single-plex ELISA for each biomarker or a multiplex ELISA.

In various embodiments, the kit does not include an ELISA for measuring concentration of TFF1, or wherein the kit does not include an assay for measuring concentrations of TFF1.

Various embodiments provide for a combination, comprising a kit of the present invention, and a urine sample. In various embodiments, the combination can further comprise a blood sample.

Various embodiments provide for a method for further confirming pancreatic ductal adenocarcinoma (PDAC) in a subject, comprising: calculating a risk score; determining the risk score to be equal or higher than a threshold score; performing one or more imaging test or one or more biopsies on the subject to further confirm the presence of PDAC. In various embodiments, the method can further comprise administering PDAC therapy to the subject.

In various embodiments, calculating the risk score can comprise performing any one of the methods described herein for calculating the risk score.

Various embodiments provide for a method of further confirming pancreatic ductal adenocarcinoma (PDAC) in a subject, and optionally treating the subject, comprising: obtaining the subject's risk score, the risk score being equal to or higher than a threshold score; and performing one or more imaging test or one or more biopsies on the subject to further confirm the presence of PDAC; and optionally administering a PDAC therapy to the subject. In various embodiments, calculating the risk score can comprise performing any one of the methods described herein for calculating the risk score.

Various embodiments provide for a method of further confirming pancreatic ductal adenocarcinoma (PDAC) in a subject, and optionally treating the subject, the method comprising: performing one or more imaging test or one or more biopsies on a subject who has been determined to have a risk score equal to or higher than a threshold score to further confirm the presence of PDAC, and optionally administering a PDAC therapy to the subject. In various embodiments, calculating the risk score can comprise performing any one of the methods described herein for calculating the risk score.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows the performance of the PancSure test. Area under the ROC curve (AUC) in the test (validation) set (50% of the samples) for the three diagnostic models: healthy versus PDAC, benign versus PDAC and pooled healthy and benign versus PDAC (for stages I-II, III-IV and I-IV) is shown.

FIG. 2 shows the violin plots that show the levels of LYVE1 and REG1B in healthy (H), benign and pancreatic ductal adenocarcinoma (PDAC) urine samples (median and interquartile ranges are indicated). The upper bars display the p-values for Kruskall-Wallis test, Dunn's multiple comparisons.

FIG. 3 shows the analytical linearity of LYVE1 and REG1B. Six urine samples combined in three mixture pools (A), (B) and (C) are analyzed. Each pool is prepared utilizing two samples, with one sample having a low biomarker value and the other sample representing a high biomarker value. Each pool is established with a highest-concentration sample, mixed with a lowest-concentration sample in five different proportions [100% (highest concentration), 75%, 50% 25% and 1%. The respective linearities for mixture pools [A/B/C] are: LYVE1 [R²=0.997/0.998/0.999] and REG1B [R²±0.987/0.99I/O.984].

FIG. 4 shows the analytical specificity of the LYVE1 and REG1B assays. Analytical specificity of the two biomarker assays was determined in urine samples by interfering substances (hemoglobin and bilirubin). Liquichek Serum Indices (Biorad, cat. no. 12012693, 12012694) are utilized. For each biomarker three urine samples are analyzed, representing (from top to bottom) low, mid, and high biomarker values. No significant interference (>20%) is observed for LYVE1 and REG1B measurements with bilirubin levels up to 23 mg/dl, and for REG1B with hemoglobin levels up to 200 mg/dl. At very high hemoglobin levels of 50 mg/dl and above, interference is observed for LYVE1 measurements: 40% at 100 mg/dl, and 93% at 200 mg/dl (indicated in red).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, NY 2006); March, Advanced Organic Chemistry Reactions, Mechanisms and Structure 7th ed., J. Wiley & Sons (New York, NY 2013); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2nd ed. (Cold Spring Harbor Press, Cold Spring Harbor NY, 2013); Kohler and Milstein, (1976) Eur. J. Immunol. 6:511; Queen et al. U.S. Pat. No. 5,585,089; and Riechmann et al., Nature 332:323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep;23 (9): 1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

LYVE1 is also known as lymphatic vessel endothelial hyaluronan receptor and extracellular link domain containing 1 (XLKD1) is a type I integral membrane glycoprotein. The encoded protein acts as a receptor and binds to both soluble and immobilized hyaluronan. References to LYVE include NCBI (GenBank) reference sequence transcript NM_006691.3 (GI: 15130120), NCBI protein ID NP_006682.2 (GI: 40549451), GeneID: 10894, and HGNC (HUGO (Human Gene Organisation) Gene Nomenclature Committee gene ID): 14687, as of Aug. 30, 2024. The gene has 4 splice variants; two are protein coding (full length protein has 322 amino acids, and the second one 218 amino acids). The full sequence of the protein is as follows:

(SEQ ID NO: 1)
MARCFSLVLLLTSIWTTRLLVQGSLRAEELSIQVSCRIMGITLVS
KKANQQLNFTEAKEACRLLGLSLAGKDQVETALKASFETCSYGWV
GDGFVVISRISPNPKCGKNGVGVLIWKVPVSRQFAAYCYNSSDTW
INSCIPEIITTKDPIFNTQTATQTTEFIVSDSTYSVASPYSTIPA
PTTTPPAPASTSIPRRKKLICVTEVFMETSTMSTETEPFVENKAA
FKNEAAGFGGVPTALLVLALLFFGAAAGLGFCYVKRYVKAFPFTN
KNQQKEMIETKVVKEEKANDSNPNEESKKTDKNPEESKSPSKTTV
RCLEAEV.

REG1B (Regenerating Family Member 1 Beta) is a protein secreted by the exocrine pancreas that is highly similar to the REGIA protein. The related REGIA protein is associated with islet cell regeneration and diabetogenesis and may be involved in pancreatic lithogenesis. References to REG1B include NCBI (GenBank) reference sequence transcript NM_006507.3 (GI: 189491779), NCBI protein ID NP_006498.1, GeneID: 5968 and HGNC: 9952 NCBI, as of Aug. 30, 2024. There are 5 splice variants of this gene (retained introns), only two code for the proteins of 166 amino acids and 149 amino acids. Protein having 166 amino acid:

(SEQ ID NO: 2)
MAQTNSFFMLISSLMFLSLSQGQESQTELPNPRISCPEGTNAYRS
YCYYFNEDPETWVDADLYCQNMNSGNLVSVLTQAEGAFVASLIKE
SSTDDSNVWIGLHDPKKNRRWHWSSGSLVSYKSWDTGSPSSANAG
YCASLTSCSGFKKWKDESCEKKFSFVCKFKN.

In recent years, many studies have been conducted with the aim to discover novel biomarkers to either replace or accompany CA19.9. Among the potential candidates, the three-biomarker panel (REG1B, LYVE1 and TFF1) with promising characteristics have been discovered by proteomic profiling of human urine and was validated in a large cohort (n=371) of control and PDAC patients using an enzyme-linked immunosorbent assay (ELISA). In a follow-up case-control study, the biomarker panel was tested in additional urine samples (n=590). In discriminating between PDAC stage I-II from healthy urines the panel achieved AUC of 0.93 in both the training (95% CI 0.903-0.969) and the validation (95% CI 0.888-0.984) datasets with sensitivity (SN) and specificity (SP)>85%. Plasma CA19.9 enhanced the performance of this panel even further, with AUC=0.99 (95% CI 0.983-1.00), SN 0.96 (95% CI 0.91-1.00), and SP =0.97 (95% CI 0.92-1.00). In analysis of benign versus PDAC I-II samples, the AUC for the combination of plasma CA19.9 and the panel increased from 0.9 to 0.92 (95% CI 0.88-0.95), with SN=0.80 (95% CI 0.71-0.88) and SP=0.89 (95% CI 0.83-0.95) when compared to plasma CA19.9 and the urinary panel alone (p<0.001 and p=0.004, respectively). Importantly, the biomarker panel combined with CA19.9 was able to detect PDAC up to 2 years prior to clinical diagnosis. Towards easier interpretation of the data, a logistic regression-based algorithm, PancRISK, was developed, which enables stratification of patients with the binary outcome: an average or an elevated risk of developing PDAC.

Based on these results and the enhanced performance of the urinary panel with CA19.9, PancSure described herein was devised as a noninvasive and rapid LDT that employs a dedicated standardized RUO kit developed by the Diagnostic Development (DxD) Hub (Singapore). TFF1 was omitted from the panel and the RUO kit was established with ELISA assays only for LYVE1 and REG1B, to be combined with blood based CA19.9 measured using already established assays in widespread clinical use. Surprisingly, the performance and predictive value of the test remain extremely high despite the removal of TFF1 as a biomarker. For test-result interpretation, PancSure utilizes a formula that combines the levels of LYVE1, REG1B, CA19.9 and age.

Described herein are analytical and clinical validation of the PancSure test in a cohort of 565 subjects in a Clinical Laboratory Improvement Amendments (CLIA)/College of American Pathologists (CAP) certified environment towards clinical validity.

As earlier detection of PDAC will likely lead to improvement in survival of patients with this malignancy, the quest for noninvasive biomarkers to this goal has fueled an active area of research. A noninvasive test has the clear advantage of being amenable to more frequent testing due to substantially reduced risks of procedural side-effects, increased patient comfort, and ease of obtaining samples more rapidly and cost-effectively. Over 4000 publications on noninvasive PDAC biomarkers have been published in the last decade, with a subset of 49 papers reporting on promising biomarker candidates. Of these, very few are close to making their transition into a viable clinical test. This is not surprising, as the development of such a test is extremely challenging. The US Early Detection Research Network (EDRN) has described the path for biomarker development and validation as a five-phase approach: (1) pre-clinical exploratory, (2) clinical grade assay development and verification, (3) retrospective longitudinal trail, (4) prospective screening trial, and (5) cancer control. Thus, developing an LDT or FDA-approvable IVD test is a time-consuming and expensive endeavor, which requires dedicated expertise and funding. Even if those prerequisites are available, many promising biomarkers fail the rigor of analytical and clinical validation steps and thus do not withstand the transition from the originating (often academic) laboratory into the clinical space. The two multi-cancer (including PDAC) early detection (MCED) blood-based tests Cancer SEEK and Grail's Galleri and PDAC-specific Bluestar Genomics blood-based tests that have made it into clinical implementation still require detailed validation of the SN (currently only at about 52-62% for early cancer stages) for early cancer detection. On the other hand, the IMMRay test (Immunovia, Lund, Sweden) targeting a panel of immune system modulators was recently withdrawn from the US market.

We describe herein the development and characterization of a PancSure—an LDT that includes ELISA detection of two urine biomarkers, REG1B and LYVE1 (and excludes TFF1). In the analytical validation of these ELISA assays, all the criteria recommended were met for precision, the inter- and intra-assay % CV are <15% and <10%, respectively; the accuracy is above 90%; and linearity is above 95% with a CV of less than 15% for at least 3 replicate measurements. As patients at various stages of PDAC may show presence of bilirubin and hemoglobin in urine, it was also crucial to determine any interference of these substances in our urine-based test. The only interference (>20%) was seen with hemoglobin at high levels of ≥50 mg/dl when measuring LYVE1, thus caution should be applied when interpreting the test results in highly hemolytic samples. With this minor exception, the devised PancSure test successfully passed all the necessary requirements and thus received approval and certification as an LDT in accordance with CLIA/CAP regulations. Phasing in the new unified ELISA kit was a necessary step in transitioning from the research-grade commercial kits to a standardized clinical assay with the objectives to secure long-term supply of raw materials and to establish quality-controlled manufacturing of the kits in sufficiently large quantities for high-volume clinical application.

For clinical interpretation of the results, a riskscore incorporates values of LYVE1, REG1B, CA19.9, as well as patient's age. While individuals with a Lewis-negative phenotype (typically around 8% of patients) will have no detectable CA19.9, as it is only one of the four variables, the risk score is still expected to produce the correct patient's stratification/classification. Current PancRISK differentiates early-stage malignancy from no-cancer controls with 80-85% SP and 82-84% SN (AUC=0.89 [0.84-0.93], 95% CI).

In various embodiments, the test described herein is intended to be used for surveillance of high-risk groups, rather than as a screening tool in the general population, as the prevalence of PDAC is too low (~12 per 100,000). In other words, PancSure, in various embodiments, is meant for risk stratification, i.e. to further enrich the population already at risk, therefore aiding the diagnosis. This aligns well with the proposed Define-Enrich-Find (DEF) approach. That said, in various embodiments of the invention, the compositions and methods is also contemplated to be used as and is used a diagnostic test for the general population even if the subject does not have any risks associated with having pancreatic cancer, or have any symptoms of pancreatic cancer.

In accordance with various embodiments, the test is used for stratification of asymptomatic individuals with genetic predisposition to PDAC development as well as in patients with symptoms suggestive of PDAC to identify those having pancreatic cancer as opposed to those who do not have pancreatic cancer. Genetic predisposition includes but are not limited to familial history, gene mutations including BRCA1/2 and PALB2; genetic syndromes-Peutz-Jeghers, Familial atypical multiple mole melanoma syndrome and Lynch syndrome., The test is based on the third predictive model which distinguished between no-cancer (healthy+benign cases) and cancer cases (including all PDAC stages).

PancSure and PancRISK are currently being externally validated in the large observational study, UroPanc (www.pcrf.org.uk/uropanc-study/, Pancreatic Cancer Research Fund UK, last access June 2023).

PancSure, described herein, presents a noninvasive, clinically certified LDT with a 48-hour turnover. As ELISA is already widely used as a platform in clinical laboratories, CA19.9 is routinely measured in the clinic,, the assay can be readily implemented without the necessity of additional and costly special instrumentation. Hence, PancSure could be offered as a viable and long-awaited test in the near future—with the potential to significantly improve earlier detection of PDAC and the care of patients with this deadly disease.

### Types of Pancreatic Cancer

The present invention is useful in the diagnosis of PDAC. The PDAC may be early stage PDAC, for example stage I or stage II PDAC, or it may be advanced-stage PDAC, for example stage III or stage IV PDAC. The present invention can be particularly useful in detecting early-stage PDAC, in particular stage I to stage IIA PDAC.

Classification of PDAC can be done according to the American Joint Committee on Cancer (AJCC) tumor-nodes-metastasis (TNM) staging system. The T score describes the size of the main (primary) tumor and whether it has grown outside the pancreas and into nearby organs. The N score describes the spread to nearby (regional) lymph nodes. The M score indicates whether the cancer has metastasized (spread) to other organs of the body:
Tx, TO, Tis: see TNM system
   T1: tumor <2 cm in greatest dimension, limited to pancreas
   T2: tumor >2 cm in greatest dimension, limited to pancreas
   T3: extension beyond pancreas, no involvement of SMA or coeliac axis
   T4: involvement of SMA or coeliac axis
Regional lymph nodes (N)
   Nx: nodes cannot be assessed
   NO: no evidence of nodal involvement
   N1: regional nodal metastases present
Metastases (M)
   Mx: presence of metastases cannot be assessed
   MO: no evidence of metastases
   M1: distant metastases present

Stage I PDAC is the earliest stage, where cancer is confined to the pancreas, and there is no cancer in the lymph nodes. In Stage II, the cancer is locally invasive. Cancer in both of these stages is still resectable; currently, fewer than 1 in 5 cancers of the pancreas (<20%) are diagnosed at stage I/II. References to stage II PDAC herein include stage IIA and IIB. In stage III, the cancer has spread beyond pancreas and is in large blood vessels, so unresectable. Stage IV cancer has metastasized to distant sites (and again not treatable by surgery). References herein to detecting or diagnosing PDAC generally refer to detecting or diagnosing each stage PDAC, in particular stage I or stage II PDAC. Such methods are particularly useful given the cancer is still treatable by resection at this stage and survival rates are much improved.

With reference to the TNM score, the stage groupings are:
stage 0: Tis NO MO
stage Ia: T1 NO MO
stage Ib: T2 NO MO
stage IIa: T3 NO MO
stage IIb: T1, T2 or T3 with N1 M0
stage III: T4 and MO (any N)
stage IV: M1 (any T any N)

### Biological Samples

The biological sample may be a urine sample, a whole blood sample, a blood-serum sample, a blood-plasma sample or a biopsy (such as a pancreatic tissue sample), although urine samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing within several hours of collection. Maintenance at −80 °C can be used for long-term storage.

The sample may be processed prior to determining the level of expression of the gene(s)/protein(s)/carbohydrate(s). The sample may be subject to enrichment (for example to increase the concentration of the biomarkers being quantified), centrifugation, or dilution. In other embodiments, the samples do not undergo any pre-processing and are used unprocessed.

In some embodiments of the invention, the biological sample may be enriched for the protein and/or carbohydrate biomarkers prior to detection and quantification (i.e. measurement). The step of enrichment can be any suitable pre-processing method step to increase the concentration of protein and/or carbohydrate biomarkers in the sample. For example, the step of enrichment may comprise centrifugation and/or filtration to remove cells or unwanted analytes from the sample.

The methods of the invention may be carried out on one test sample from a patient. Alternatively, more than one sample may be taken from a patient, for example 2, 3, 4 or 5 samples. Each sample may be subjected to a single assay to quantify one of the protein and/or carbohydrate biomarkers, or alternatively a sample may be tested for all of the protein and/or carbohydrate biomarkers being quantified.

Methods for detecting the levels of protein expression include any methods known in the art. Examples include but are not limited to indirectly measuring protein expression using DNA or mRNA arrays, directly measuring protein expression by measuring the level of protein synthesis or measuring protein concentration.

Protein microarrays can also be used to directly detect protein expression. They can comprise capture molecules fixed to a solid surface. Capture molecules are most commonly antibodies specific to the proteins being detected. Further capture molecules include proteins, aptamers, nucleic acids, receptors and enzymes. Accordingly, embodiments of the invention include a protein microarray comprising capture molecules (such as antibodies) specific for each of the protein and/or carbohydrate biomarkers being quantified immobilised on a solid support. In one embodiment of the invention, the microarray comprises capture molecules specific for each of LYVE1 protein, REG1B protein, and CA 19.9 carbohydrate.

Once captured on a microarray, detection methods can be any of those known in the art. For example, fluorescence detection can be employed. Other detection methods include other optical methods (for example colorimetric analysis, chemiluminescence, label free Surface Plasmon Resonance analysis, microscopy, reflectance etc.), mass spectrometry, electrochemical methods (for example voltammetry and amperometry methods) and radio frequency methods (for example multipolar resonance spectroscopy).

Additional methods of determining protein concentration include mass spectrometry and/or liquid chromatography, such as LC-MS, UPLC, or a tandem UPLC-MS/MS system.

In those embodiments of the invention in which the binding molecule is an antibody or antibody fragment, the method of the invention can be performed using any immunological technique known in the art. For example, ELISA, radio immunoassays or similar techniques may be utilised. Another method for detecting the complex may utilise antibodies or antigens that have been labelled with radioisotopes followed by a measure of radioactivity. Examples of radioactive labels for antigens include ³H, ¹⁴C and ¹²⁵I.

Various embodiments of the invention provide for significantly improving earlier detection of PDAC and the care of patients with this deadly disease.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula I:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula I ]$$

wherein Lis log_{c}(concentration of LYVE1+0.01), R is log_{c}(concentration REG1B+0.01), C is log_{c}(concentration CA19.9-+0.01), and A is age of subject, and
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein β0 is the constant (independent variable), and β1, β2, β3, β4 are the dependent variables: β1 is the standardized weight for LYVE1, β2 is the standardized weight for REG1B, β3 is the standardized weight for CA19.9 and β4 is the standardized weight for age.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ia:

$$\frac{( 1 )}{( 1 + {e}^{( - ( - 6.895079 - ( 0.007487015 * L ) + ( 0.3566643 * R ) + ( 0.7100223 * C ) + ( 0.0399479 * A ) ) )} )} , \qquad [ Formula Ia ]$$

wherein L is log_{c}(concentration of LYVE1+0.01), R is log_{c}(concentration REG1B+0.01), C is log_{c}(concentration CA19.9 protein+0.01), and A is age of subject.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ib1:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula Ib1 ]$$

wherein L is log_{c}(concentration of LYVE1+0.01), R is log_{c}(concentration REG1B+0.01), C is log_{c}(concentration CA19.9 protein+0.01), and A is age of subject; and
wherein
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein
β0 is −6.895079±0.5,
β1 is 0.007487015±0.001,
β2 is 0.3566643±0.1,
β3 is 0.7100223±0.1, and
β4 is 0.0399479±0.01.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ib2:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula Ib2 ]$$

wherein L is log_{c}(concentration of LYVE1±0.01), R is log_{c}(concentration REG1B+0.01), C is log_{c}(concentration CA19.9 protein+0.01), and A is age of subject; and
wherein
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein
β0 is −6.895079±0.75,
β1 is 0.007487015±0.005,
β2 is 0.3566643±0.5,
β3 is 0.7100223±0.5, and
β4 is 0.0399479±0.05.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ib3:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula Ib3 ]$$

wherein L is log_{c}(concentration of LYVE1±0.01), R is log_{c}(concentration REG1B+0.01), C is log_{c}(concentration CA19.9 protein+0.01), and A is age of subject; and
wherein
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein
β0 is −6.90,
β1 is 0.007,
β2 is 0.36,
β3 is 0.71, and
β4 is 0.04.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ib4:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula Ib4 ]$$

wherein L is log_{c}(concentration of LYVE1±0.01), R is log_{c}(concentration REG1B+0.01), C is log_{c}(concentration CA19.9 protein+0.01), and A is age of subject; and
wherein
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein
β0 is −6.90±0.5,
β1 is 0.007±0.001,
β2 is 0.36±0.1,
β3 is 0.71±0.1, and
β4 is 0.04±0.01.

Various embodiments of the invention provide for a method of calculating a risk score for a subject, comprising: obtaining urine concentrations of lymphatic vessel endothelial hyaluronan receptor 1 (LYVE1) protein, and regenerating islet-derived 1B (REG1B) protein; obtaining the subject's age; obtaining blood concentration of CA19.9 carbohydrate; and calculating the risk score using the following Formula Ib5:

$$\frac{1}{( 1 + {e}^{( - ( β 0 ) + ( β 1 * L ) + ( β 2 * R ) + ( β 3 * C ) + ( β 4 * A ) ) )} )} , \qquad [ Formula Ib5 ]$$

wherein L is log_{c}(concentration of LYVE1±0.01), R is log_{c}(concentration REG1B+0.01), C is log_{c}(concentration CA19.9 protein+0.01), and A is age of subject; and
wherein
wherein the calculation for each β is: β=b*SD_{IV}*R/SD_{LogitY} and wherein
β is the standardized weight,
b is the unstandardized weight,
SD_{IV} is the standard deviation of the independent variable also known as the predictor,
R is the Pearson correlation coefficient (obtained from the logistic regression), and SD_{LogitY} is the standard deviation of the log odds predicted probability; and
wherein
β0 is −6.90±0.75,
β1 is 0.007±0.005,
β2 is 0.36±0.5,
β3 is 0.71±0.5, and
β4 is 0.04±0.05.

In various embodiments, the method further comprises: comparing the risk score to a threshold score; and stratifying a subject as having pancreatic cancer if the risk score is equal to or higher than the threshold score, or stratifying a subject as not having pancreatic cancer if the risk score is lower than the threshold score.

In various embodiments, stratifying a subject as having pancreatic cancer comprises any one of the specificities, sensitivities, or both as set forth in Table 8. In various embodiments, stratifying a subject as having pancreatic cancer comprises any one of the positive predictive values (PPV) as set forth in Table 9.

In various embodiments, the subject has one or more symptoms of pancreatic cancer. Symptoms of pancreatic cancer include but are not limited to Abdominal pain (non-specific), Benign biliary stricture, Pancreatitis (chronic), Pancreatitis (recurrent acute), Pancreatitis (autoimmune), Pancreatitis (idiopathic), Cholecystitis, Choledocholiathiasis, Cholelithiasis, Dilated pancreatic duct, Pancreatic cysts and pseudocysts, Intraductal Papillary Mucinous Neoplasm (IPMN), Cystadenoma. Additional examples of symptoms include but are not limited to abdominal pain that spreads to the sides or back, loss of appetite, weight loss, yellowing of the skin and the whites of the eyes (jaundice), light-colored or floating stools, dark-colored urine, itching, new diagnosis of diabetes or diabetes that is getting harder to control, pain and swelling in an arm or leg (that might be caused by a blood clot), and tiredness or weakness.

In various embodiments the subject has one or more genetic predispositions to PDAC development. Examples of these genetic predispositions include but are not limited to familial history, gene mutations including BRCA1/2 and PALB2; genetic syndromes-Peutz-Jeghers, Familial atypical multiple mole melanoma syndrome and Lynch syndrome.

In various embodiments, the threshold score is 0.412. In various embodiments, the threshold score is 0.38, 0.39, 0.40, 0.41, 0.42, 0.43, 0.44, or 0.45. In various embodiments, the threshold score is 0.407, 0.408, 0.409, 0.410, 0.411, 0.412, 0.413, 0.414, 0.415, 0.416, or 0.147. In various embodiments, the threshold score is calculated from LYVE1 protein concentration, REG1B concentration, subject's age; and concentration of CA19.9 carbohydrate from healthy subjects. In various embodiments, the threshold score is calculated from LYVE1 protein concentration, REG1B concentration, subject's age; and concentration of CA19.9 carbohydrate from pancreatic cancer subjects. In various embodiments, the threshold score is calculated from LYVE1 protein concentration, REG1B concentration, subject's age; and concentration of CA19.9 carbohydrate from both healthy subjects and pancreatic cancer subjects.

In various embodiments, the method does not include obtaining concentrations of TFF1, In various embodiments, the method does not include obtaining urine concentrations of TFF1.

In various embodiments, obtaining urine concentrations of LYVE1 and REG1B comprises: contacting a urine sample, obtained from the subject, to an assay, the assay comprising: one enzyme-linked immunosorbent assay (ELISA) for measuring the concentrations of LYVE1 and REG1B, or two ELISAs, a first ELISA for measuring the concentration of LYVE1 and a second ELISA for measuring the concentration of REG1B.

In various embodiments, the one ELISA comprises an antibody that specifically binds to LYVE1 and an antibody that specifically binds to REG1B, or the two ELISAs comprise the first ELISA comprising an antibody that specifically binds to LYVE1 and the second ELISA comprising an antibody that specifically binds to REG1B.

In various embodiments, the one ELISA comprises a primary capture antibody that specifically binds to LYVE1 and a primary capture antibody that specifically binds to REG1B, or the two ELISAs comprise the first ELISA comprising a primary capture antibody that specifically binds to LYVE1 and the second ELISA comprising a primary capture antibody that specifically binds to REG1B.

In various embodiments, the one ELISA comprises a secondary detection antibody pair that specifically binds to LYVE1 including an additional antibody conjugated to a reporter molecule and a secondary detection antibody pre-conjugated with a reporter molecule that specifically binds to REG1B, or the two ELISAs comprise the first ELISA comprising a secondary detection antibody pair that specifically binds to LYVE1 including an additional antibody conjugated to a reporter molecule and the second ELISA comprising a secondary detection antibody pre-conjugated with a reporter molecule that specifically binds to REG1B.

In various embodiments, obtaining urine concentrations of LYVE1 and REG1B comprises using an antibody that specifically binds to LYVE1 and an antibody that specifically binds to REG1B.

In various embodiments, obtaining urine concentrations of LYVE1 and REG1B comprises using a primary capture antibody that specifically binds to LYVE1 and a primary capture antibody that specifically binds to REG1B.

In various embodiments, obtaining urine concentrations of LYVE1 and REG1B comprises using a secondary detection antibody pair that specifically binds to LYVE1 including an additional antibody conjugated to a reporter molecule and a secondary detection antibody pre-conjugated with a reporter molecule that specifically binds to REG1B.

In various embodiments, the urine sample or blood sample is obtained from a human subject having or suspected of having pancreatic ductal adenocarcinoma (PDAC), In various embodiments, the urine sample or blood sample is obtained from a human subject having one or more genetic predispositions to PDAC development. In various embodiments, the urine sample or blood sample is obtained from a human subject having one or more symptoms of pancreatic cancer. In various embodiments, the pancreatic ductal adenocarcinoma is stage I, stage II, stage III or stage IV pancreatic ductal adenocarcinoma. In various embodiments, the urine or blood sample is obtained from a human subject with no symptoms of having or suspected of having PDAC, no genetic predispositions to PDAC development, or both.

In various embodiments, obtaining blood concentration of CA19.9 carbohydrate comprises contacting a blood sample, obtained from the subject, to an enzyme-linked immunosorbent assay (ELISA) or a chemiluminescence assay for measuring the concentration of CA19.9 carbohydrate.

In various embodiments, obtaining blood concentration of CA19.9 protein comprises using an immunoassay.

In various embodiments, the method does not include obtaining concentrations of TFF1. In various embodiments, the method does not include obtaining urine concentrations of TFF1.

In various embodiments, the subject does not have a serum hemoglobin concentration of ≥50 mg/dl, particularly when measuring LYVE1 in urine.

In various embodiments, the method comprises stratifying the subject as having pancreatic cancer if the risk score is equal to or higher than the threshold score and performing an imaging test, a biopsy, or both on the subject to further confirm the presence of PDAC. Examples of imaging tests include but are not limited to computed tomography (CT) scan (e.g., multiphase CT scan or a pancreatic protocol CT scan), magnetic resonance imaging (MRI) (e.g., MR cholangiopancreatography (MRCP), MR angiography (MRA)), ultrasound (US) (e.g., Abdominal ultrasound, Endoscopic ultrasound (EUS)), cholangiopancreatography (e.g., Endoscopic retrograde cholangiopancreatography (ERCP), Magnetic resonance cholangiopancreatography (MRCP), Percutaneous transhepatic cholangiography (PTC)), positron emission tomography (PET) scan. Examples of biopsies include but are not limited to fine needle aspiration (FNA), endoscopic biopsy (e.g., endoscopic ultrasound (EUS) to pass a needle into the tumor or endoscopic retrograde cholangiopancreatography (ERCP) to place a brush to remove cells from the bile or pancreatic ducts), surgical biopsy. Thereafter, the method can further comprise administering PDAC therapy to the subject.

In various embodiments, the method comprises stratifying a subject as having pancreatic cancer if the risk score is equal to or higher than the threshold score and further comprising administering PDAC therapy to the subject.

In various embodiments, the method further comprises administering PDAC therapy to the subject.

PDAC therapy (in particular stage I and stage II PDAC) can involve resecting the tumor. PDAC therapy may alternatively or additionally involve treatment by chemotherapy and/or radiotherapy. Treatment by chemotherapy may include administration of gemcitabine and/or Folfirinox. Folfirinox is a combination of fluorouracil (5-FU), irinotecan, oxaliplatin and folinic acid (leucovorin). Treatment regimens involving Folfirinox may comprise administration of oxaliplatin, followed by folinic acid, followed by irinotecan (alternatively irinotecan may be administered at the same time as folinic acid), followed by 5-FU.

In various embodiments, the method comprises stratifying a subject as not having pancreatic cancer if the risk score is lower than the threshold score and the method further comprises monitoring the subject for PDAC. In various embodiments, monitoring the subject for PDAC comprises repeating any one of the methods of calculating a risk score for a subject as described herein. In various embodiments, repeating any one of the methods of calculating a risk score for a subject as described herein comprises repeating the method every about 3-6 months. In various embodiments, repeating any one of the methods of calculating a risk score for a subject as described herein comprises repeating the method every about 6-9 months. In various embodiments, repeating any one of the methods of calculating a risk score for a subject as described herein comprises repeating the method every about 12 months. In various embodiments, repeating any one of the methods of calculating a risk score for a subject as described herein comprises repeating the method every about 18 months. In various embodiments, repeating any one of the methods of calculating a risk score for a subject as described herein comprises repeating the method every about 2 years, about 3 years, about 4 years, or about 5 years. In various embodiments, repeating any one of the methods of calculating a risk score for a subject as described herein comprises repeating the method every about 10 years.

In various embodiments, monitoring the subject for PDAC comprises periodically performing a non-invasive pancreatic cancer test. In various embodiments, the periodic interval is about every 3-6 months, about 6-9 months, about 9-12 months, about every 12 months, about every 18 months, about every 2 years, about every 3 years, about every 4 years, about every 5 years, or about every 10 years. Examples of non-invasive tests include but are not limited to blood tests (e.g., CA 19-9, CEA, liver function tests), stool examination (e.g., for blood or abnormal cells) and genetic testing.

Various embodiments of the invention provide for a method of testing a subject in need thereof, comprising: detecting concentration of LYVE1 protein in a urine sample and comparing the LYVE1 protein concentration to a reference range for a healthy subject, or comparing the LYVE1 protein concentration to a biological range for a cancer subject; detecting concentration of REG1B protein in a urine sample and comparing the REG1B protein concentration to a reference range for a healthy subject, or comparing the REG1B protein concentration to a biological range for a cancer subject; detecting concentration of CA19.9 carbohydrate in a blood sample; and obtaining the subject's age.

In various embodiments, the method further comprises detecting the high likelihood of the subject having PDAC when the subject's LYVE1 protein concentration is within the biological range for a cancer subject, and REG1B protein concentration is within the biological range for a cancer subject. In various embodiments, the method further comprises performing one or more imaging tests, one or more biopsies, or both on the subject detected to have a high likelihood of having PDAC.

In various embodiments, a reference range for LYVE1 in a healthy subject is 1.56 ng/ml-79.63 ng/ml. In various embodiments, a reference range for REG1B in a healthy subject is 7.81 ng/ml-97.93 ng/ml.

In various embodiments, a biological range for LYVE1 in a cancer subject is 3.59
100.00 ng/ml. In various embodiments, a biological range for REG1B in a cancer subject is 7.81
500.00 ng/ml.

In various embodiments, a reportable range for LYVE1 is 1.56 ng/ml-100.00 ng/ml. In various embodiments, a reportable range for REG1B is 7.81 ng/ml-500 ng/ml.

In various embodiments, obtaining urine concentrations of LYVE1 and REG1B comprises: contacting a urine sample, obtained from the subject, to an assay, the assay comprising: one enzyme-linked immunosorbent assay (ELISA) for measuring the concentrations of LYVE1 and REG1B, or two ELISAs, a first ELISA for measuring the concentration of LYVE1 and a second ELISA for measuring the concentration of REG1B.

In various embodiments, the one ELISA comprises an antibody that specifically binds to LYVE1 and an antibody that specifically binds to REG1B, or the two ELISAs comprise the first ELISA comprising an antibody that specifically binds to LYVE1 and the second ELISA comprising an antibody that specifically binds to REG1B.

In various embodiments, obtaining urine concentrations of LYVE1 and REG1B comprises using an antibody that specifically binds to LYVE1 and an antibody that specifically binds to REG1B.

In various embodiments, obtaining urine concentrations of LYVE1 and REG1B comprises using a primary capture antibody that specifically binds to LYVE1 and a primary capture antibody that specifically binds to REG1B.

In various embodiments, obtaining urine concentrations of LYVE1 and REG1B comprises using a secondary detection antibody pair that specifically binds to LYVE1 including an additional antibody conjugated to a reporter molecule and a secondary detection antibody pre-conjugated with a reporter molecule that specifically binds to REG1B.

In various embodiments, the urine sample or blood sample is obtained from a human subject having or suspected of having pancreatic ductal adenocarcinoma (PDAC), In various embodiments, the urine sample or blood sample is obtained from a human subject having one or more genetic predispositions to PDAC development. In various embodiments, the urine sample or blood sample is obtained from a human subject having one or more symptoms of pancreatic cancer. In various embodiments, the urine or blood sample is obtained from a human subject with no symptoms of having or suspected of having PDAC, no genetic predispositions to PDAC development, or both.

In various embodiments, the pancreatic ductal adenocarcinoma is stage I, stage II, stage III or stage IV pancreatic ductal adenocarcinoma.

In various embodiments, obtaining blood concentration of CA19.9 carbohydrate comprises contacting a blood sample, obtained from the subject, to an enzyme-linked immunosorbent assay (ELISA) or a chemiluminescence assay for measuring the concentration of CA19.9 carbohydrate. In various embodiments, obtaining blood concentration of CA19.9 protein comprises using an immunoassay.

In various embodiments, the method does not include obtaining concentrations of TFF1. In various embodiments, the method does not include obtaining urine concentrations of TFF1.

In various embodiments the method further comprises performing an imaging test, a biopsy, or both on the subject who has been detected to have a high likelihood of having PDAC to further confirm the presence of PDAC. Examples of imaging tests include but are not limited to computed tomography (CT) scan (e.g., multiphase CT scan or a pancreatic protocol CT scan), magnetic resonance imaging (MRI) (e.g., MR cholangiopancreatography (MRCP), MR angiography (MRA)), ultrasound (US) (e.g., Abdominal ultrasound, Endoscopic ultrasound (EUS)), cholangiopancreatography (e.g., Endoscopic retrograde cholangiopancreatography (ERCP), Magnetic resonance cholangiopancreatography (MRCP), Percutaneous transhepatic cholangiography (PTC)), positron emission tomography (PET) scan. Examples of biopsies include but are not limited to fine needle aspiration (FNA), endoscopic biopsy (e.g., endoscopic ultrasound (EUS) to pass a needle into the tumor or endoscopic retrograde cholangiopancreatography (ERCP) to place a brush to remove cells from the bile or pancreatic ducts), surgical biopsy. Thereafter, the method can further comprise administering PDAC therapy to the subject.

In various embodiments, the method further comprises comprising administering PDAC therapy to the subject.

PDAC therapy (in particular stage I and stage II PDAC) can involve resecting the tumor. PDAC therapy may alternatively or additionally involve treatment by chemotherapy and/or radiotherapy. Treatment by chemotherapy may include administration of gemcitabine and/or Folfirinox. Folfirinox is a combination of fluorouracil (5-FU), irinotecan, oxaliplatin and folinic acid (leucovorin). Treatment regimens involving Folfirinox may comprise administration of oxaliplatin, followed by folinic acid, followed by irinotecan (alternatively irinotecan may be administered at the same time as folinic acid), followed by 5-FU.

In various embodiments, when the subject does not have a high risk of having PDAC, the method further comprises monitoring the subject for PDAC. In various embodiments, monitoring the subject for PDAC comprises repeating any one of the methods of calculating a risk score for a subject as described herein. In various embodiments, repeating any one of the methods of calculating a risk score for a subject as described herein comprises repeating the method every about 3-6 months. In various embodiments, repeating any one of the methods of calculating a risk score for a subject as described herein comprises repeating the method every about 6-9 months. In various embodiments, repeating any one of the methods of calculating a risk score for a subject as described herein comprises repeating the method every about 12 months. In various embodiments, repeating any one of the methods of calculating a risk score for a subject as described herein comprises repeating the method every about 18 months. In various embodiments, repeating any one of the methods of calculating a risk score for a subject as described herein comprises repeating the method every about 2 years, about 3 years, about 4 years, or about 5 years. In various embodiments, repeating any one of the methods of calculating a risk score for a subject as described herein comprises repeating the method every about 10 years.

In various embodiments, monitoring the subject for PDAC comprises periodically performing a non-invasive pancreatic cancer test. In various embodiments, the periodic interval is about every 3-6 months, about 6-9 months, about 9-12 months, about every 12 months, about every 18 months, about every 2 years, about every 3 years, about every 4 years, about every 5 years, or about every 10 years. Examples of non-invasive tests include but are not limited to blood tests (e.g., CA 19-9, CEA, liver function tests), stool examination (e.g., for blood or abnormal cells) and genetic testing.

Various embodiments provide for a method for further confirming the presence of pancreatic ductal adenocarcinoma (PDAC) in a subject, and optionally treating the subject, comprising: calculating a risk score; determining the risk score to be equal to or higher than a threshold score; and performing an imaging test, a biopsy, or both on the subject to further confirm the presence of PDAC. In various embodiments, the method further comprises administering PDAC therapy to the subject. In various embodiments, calculating the risk score comprising performing any of the methods described herein. The threshold score can also be any one of the threshold scores as described herein.

Various embodiments provide for a method for further confirming the presence of pancreatic ductal adenocarcinoma (PDAC) in a subject, and optionally treating the subject, comprising: obtaining the subject's risk score, the risk score being equal to or higher than a threshold score; and performing an imaging test, a biopsy, or both on the subject to further confirm the presence of PDAC. In various embodiments, the method further comprises administering a PDAC therapy to the subject. In various embodiments, calculating the risk score comprising performing any of the methods described herein. The threshold score can also be any one of the threshold scores as described herein.

Various embodiments provide for a method for treating pancreatic ductal adenocarcinoma (PDAC) in a subject, comprising: administering a PDAC therapy to a subject who has been determined to have a risk score equal to or higher than a threshold risk score. In various embodiments, calculating the risk score comprising performing any of the methods described herein. The threshold score can also be any one of the threshold scores as described herein.

Various embodiments provide for a method for further confirming pancreatic ductal adenocarcinoma (PDAC) in a subject and optionally treating the subject, comprising: detecting the high likelihood of the subject having PDAC by performing any one of the methods of the present invention described herein; performing an imaging test, a biopsy, or both on the subject to further confirm the presence of PDAC; and optionally, administering PDAC therapy to the subject. In various embodiments, the method comprises administering PDAC therapy to the subject.

Various embodiments provide for a method for further confirming pancreatic ductal adenocarcinoma (PDAC) in a subject, comprising: performing an imaging test, a biopsy, or both on the subject to further confirm the presence of PDAC in a subject who has been detected to have a high likelihood of having PDAC, wherein the subject has been detected to have the high likelihood of having PDAC by performing any one of the methods of the present invention described herein. In various embodiments, the method further comprising administering a PDAC therapy to the subject confirmed to have PDAC.

Various embodiments provide for a method for treating pancreatic ductal adenocarcinoma (PDAC) in a subject, comprising: administering a PDAC therapy to a subject who has been detected to have a high likelihood having PDAC, wherein the subject has been detected to the high likelihood of having PDAC by performing any one of the methods of the present invention described herein.

Examples of imaging tests include but are not limited to computed tomography (CT) scan (e.g., multiphase CT scan or a pancreatic protocol CT scan), magnetic resonance imaging (MRI) (e.g., MR cholangiopancreatography (MRCP), MR angiography (MRA)), ultrasound (US) (e.g., Abdominal ultrasound, Endoscopic ultrasound (EUS)), cholangiopancreatography (e.g., Endoscopic retrograde cholangiopancreatography (ERCP), Magnetic resonance cholangiopancreatography (MRCP), Percutaneous transhepatic cholangiography (PTC)), positron emission tomography (PET) scan. Examples of biopsies include but are not limited to fine needle aspiration (FNA), endoscopic biopsy (e.g., endoscopic ultrasound (EUS) to pass a needle into the tumor or endoscopic retrograde cholangiopancreatography (ERCP) to place a brush to remove cells from the bile or pancreatic ducts), surgical biopsy. Thereafter, the method can further comprise administering PDAC therapy to the subject.

Treatment for PDAC (in particular stage I and stage II PDAC) can involve resecting the tumor. Treatment may alternatively or additionally involve treatment by chemotherapy and/or radiotherapy. Treatment by chemotherapy may include administration of gemcitabine and/or Folfirinox. Folfirinox is a combination of fluorouracil (5-FU), irinotecan, oxaliplatin and folinic acid (leucovorin). Treatment regimens involving Folfirinox may comprise administration of oxaliplatin, followed by folinic acid, followed by irinotecan (alternatively irinotecan may be administered at the same time as folinic acid), followed by 5-FU.

Other embodiments of the invention provide for a method of diagnosing PDAC comprising contacting a biological sample (such as a urine sample) from a patient with reagents or binding molecules specific for the biomarker proteins and/or carbohydrate being quantified, and measuring the abundance of protein-reagent or protein-binding molecule complexes, and/or carbohydrate-reagent or carbohydrate-binding molecule complexes, and correlating the abundance of protein-reagent or protein-binding molecule complexes and/or carbohydrate-reagent or carbohydrate-binding molecule complexes, with the concentration of the relevant protein in the biological sample. For example, in one embodiments of the invention, the method comprises the steps of:
(a) contacting a biological sample (such as a urine sample and a blood sample) with reagents or binding molecules specific for one or more of LYVE1, REG1B and CA19.9;
(b) quantifying the abundance of protein-reagent or protein-binding molecule and carbohydrate-reagent or carbohydrate-binding molecule complexes for one or more of LYVE1, REG1B and CA19.9; and
(c) correlating the abundance of protein-reagent or protein-binding molecule and carbohydrate-reagent or carbohydrate-binding molecule complexes with the concentration of one or more of the proteins LYVE1 and REG1B, as well as of the carbohydrate CA19.9 in the biological sample.

The method may further comprise the step of d) comparing the concentration of the proteins and carbohydrate in step c) with a reference to determine the presence or absence of PDAC. The patient can then be treated accordingly.

In various embodiments, the method excludes the detection of TFF1.

In some embodiments of the invention, the methods comprise contacting the biological sample with reagents or binding molecules specific for one, two or three of LYVE1, REG1B and CA19.9. In various embodiments, the method does not include use of reagents or binding for TFF1. Suitable reagents or binding molecules may include an antibody or antibody fragment, an enzyme, a nucleic acid, an organelle, a cell, a biological tissue, imprinted molecule or a small molecule. Such methods may be carried out using kits or biosensors of the invention.

The present invention also provides a method of diagnosis for pancreatic ductal adenocarcinoma comprising detecting concentration of LYVE1 and REG1B proteins and CA19.9 carbohydrate, in one or more biological samples, in particular a urine sample and blood sample. In various embodiments, the method excludes the detecting the concentration of TFF1 in the one or more biological samples.

The presence of pancreatic ductal adenocarcinoma can be determined by detecting an increase in protein and carbohydrate concentration as compared with the concentration of the corresponding proteins and CA19.9 in samples taken from healthy control subjects.

### KITS

The present invention is also directed to a kit to detect PDAC, or to calculate a PDAC risk score. The kit is useful for practicing the inventive method of detecting PDAC, or calculating a PDAC risk score. The kit is an assemblage of materials or components, including at least one of the inventive compositions. Thus, in some embodiments the kit includes ELISA(s) having immobilized capture antibodies and secondary detection antibody pairs, as described above.

In various embodiments, the exact nature of the components configured in the inventive kit is configured particularly for the purpose of screening human subjects.

Instructions for use may be included in the kit. “Instructions for use” typically include a tangible expression describing the technique to be employed in using the components of the kit to effectuate a desired outcome, such as to detect PDAC, or to calculate a PDAC risk score. Optionally, the kit also contains other useful components, such as, diluents, buffers, enzymes, antibodies, surface-immobilized targeted LYVE1 and REG1B proteins, reporter molecules, reaction stop solutions, disposable materials including reaction chambers (microwells and/or microtiter plates), pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in ELISA kits. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

Various embodiments provide for a kit for testing for pancreatic ductal adenocarcinoma comprising: one enzyme-linked immunosorbent assay (ELISA) for measuring the concentration of Lymphatic vessel endothelial hyaluronan receptor 1 (LYVE1) and Regenerating Family Member 1 Beta (REG1B), or two ELISAs, a first ELISA for measuring concentration of LYVE1, and a second ELISA for measuring concentration of REG1B.

Various embodiments provide for a kit for stratifying the risk for pancreatic ductal adenocarcinoma comprising: one enzyme-linked immunosorbent assay (ELISA) for measuring the concentration of Lymphatic vessel endothelial hyaluronan receptor 1 (LYVE1) and Regenerating Family Member 1 Beta (REG1B), or two ELISAs, a first ELISA for measuring concentration of LYVE1, and a second ELISA for measuring concentration of REG1B.

In various embodiments, the kit further comprises one chemiluminescence assay for measuring the concentration CA19.9 carbohydrate.

In various embodiments, the kit further comprises one or more solvents for diluting

LYVE1 and REG1B proteins or protein concentrations in a urine sample.

In various embodiments, the kit further comprises one or more solvents for extracting LYVE1 and REG1B proteins from a urine sample.

In various embodiments, antibodies specific to LYVE1 and REG1B are immobilized on a solid surface. In various embodiments, antibodies specific to LYVE1 and REG1B are immobilized on a microtiter plate.

In various embodiments, the ELISA is a single-plex ELISA for each biomarker or a multiplex ELISA.

In various embodiments, the ELISA is a single-plex ELISA for LYVE1 and a single-plex ELISA for REG1B.

In various embodiments, the ELISA is a multiplex ELISA for LYVE1 and for REG1B.

In various embodiments, the kit does not include an ELISA for measuring concentration of TFF1, or wherein the kit does not include an assay for measuring concentrations of TFF1.

Various embodiments provide for a combination, comprising a kit of the present invention (as described herein) and a urine sample. In various embodiments, the combination further comprises a blood sample. In various embodiments, the urine sample or blood sample is obtained from a human subject having or suspected of having pancreatic ductal adenocarcinoma (PDAC), In various embodiments, the urine sample or blood sample is obtained from a human subject having one or more genetic predispositions to PDAC development. In various embodiments, the urine sample or blood sample is obtained from a human subject having one or more symptoms of pancreatic cancer. In various embodiments, the urine or blood sample is obtained from a human subject with no symptoms of having or suspected of having PDAC, no genetic predispositions to PDAC development, or both.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Materials and Methods

### Clinical Samples

Urine and plasma samples utilized in this study were collected at Queen Mary University London (QMUL) and the Cedars-Sinai Medical Center using the shared standardized operating procedures: all samples were collected prior surgery or any cancer treatment and were frozen and stored at −80 °C within a four-hour window post collection. 565 urine specimens (117 healthy, 242 benign and 206 PDAC) were analyzed. Healthy cases included nine cases with familial history (FH) of PDAC. Benign (symptomatic) samples included 136 specimens collected from patients with pancreatitis, 73 with pancreatic cysts and 33 with other benign pancreatic diseases such as choledocholithiasis, biliary strictures, dilated pancreatic ducts, etc., as detailed in Table 1. Thus, the number of high-risk cases (FH, pancreatitis and benign cysts) in this study totals 218 of 565 samples and constitutes around 39% of the cohort. PDAC cases were represented by 161 stages I-II and 45 stages III-IV. Details of study groups, sample composition and patient demographics are provided in Table 2. Clinical specimens were obtained after patients consent and with the approval of the respective institutional review boards: 05/Q0408/65 for QMUL and Pro00041571 for Cedars-Sinai samples.

TABLE 1
Benign samples analyzed in the study.

| | |
|---|---|
| Abdominal pain (non-specific) | 1 |
| Benign biliary stricture | 2 |
| Other benign pancreatic diseases | 15 |
| Pancreatitis (chronic) | 122 |
| Pancreatitis (recurrent acute) | 9 |
| Pancreatitis (autoimmune) | 3 |
| Pancreatitis (idiopathic) | 2 |
| Cholecystitis | 5 |
| Choledocholiathiasis | 2 |
| Cholelithiasis | 1 |
| Dilated pancreatic duct | 7 |
| Non-neoplastic: benign cysts (39) and pseudocysts (3) | 42 |
| Neoplastic cysts: IPMN (19) plus Cystadenoma (12) | 31 |
| All benign cases | 242 |

TABLE 2
Demographics of pancreatic ductal adenocarcinoma (PDAC) patients and controls.

| | Healthy controls | | | Benign controls | | | PDAC | | |
|---|---|---|---|---|---|---|---|---|---|
| | | Age range | | | Age range | | | Age range | |
| Sex | Sample | in years | Sex | Sample | in years | Sex | Sample | in years | Sex |
| (n) | (n) | (average) | (n) | (n) | (average) | (n) | (n) | (average) | (n) |
| F = 305 | 117 | 23-83 (52) | F = 68 | 242 | 20-91 (60) | F = 125 | 206 | 20-90 (67) | F = 112 |
| (54%) | | | (58%) | | | (52%) | | | (54%) |
| M = 260 | | | M = 49 | | | M = 117 | | | M = 94 |
| (46%) | | | (42%) | | | (48%) | | | (46%) |

### Immunoassays

Proprietary ELISA kits for LYVE1 and REG1B LDT were developed and manufactured by Diagnostic Development Hub (DxD Hub, Singapore, Singapore) and were utilized in all analytical and clinical performance studies. The assays measure the respective target analyte in human urine using a 96-well microplate ELISA, with separate plates for each biomarker. The REG1B assay utilizes an anti-human REG1B monoclonal primary antibody for target capture and a secondary monoclonal antibody pre-conjugated with HRP for detection (one-step sandwich ELISA). The LYVE1 assay is a two-step sandwich ELISA, which employs a monoclonal primary and secondary polyclonal antibody pair, with an additional streptavidin-HRP binding to biotin-labeled secondary antibody.

The absorbance was measured at 450 nm using a 96-well microplate spectrophotometer (Synergy HTX, BioTek, Winooski, VT). For both assays the plate wells were pre-coated with the capture (primary) antibody. Commercially available R&D Systems Human LYVE-1 DuoSet ELISA (cat. no. DY2089, R&D Systems, Minneapolis, MN), and Sino Biological Human REG1B ELISA Kit (cat. no. SEK11638, Wayne, PA) were used as third-party comparators in the analytical accuracy studies, following the manufacturer's protocols. All samples were tested in duplicates.

Plasma CA19.9 was measured by standard chemiluminescence assays using both Roche's Cobas 601E (ECLIA) platforms with the Elecsys CA19.9 kit (Cat #11776193 122, Roche Diagnostics, Indianapolis, IN), as well as the Abbott Architect c16000 with the CA 19-9XR kit (Des Plaines, IL).

### Data Analysis

Quantitation of the biomarkers levels was extrapolated from respective standard curves for each biomarker using a 4-parameter logistic (4PL) regression curve algorithm provided by MyAssays freeware (htwww.myassays.com/four-parameter-logistic-curve.assay, last access July 2022). The software allows for transferring and displaying test results (analyte concentration for all samples) in an Excel sheet.

PancRISK was developed as described previously. Patient age and CA19-9 values were used as continuous variables, while protein concentration data were natural-log-transformed and mean-centered prior to the analysis. The biomarker panel was investigated for its ability to discriminate between experimental groups using a receiver operating characteristics (ROC) curve analysis approach. Internal validation was performed by splitting the whole dataset into the training and validation sets in a 1:1 ratio (50:50 random split at seed 111). Logistic regression was applied with a first analysis comprising healthy and PDAC samples, a second one including benign and PDAC samples, and a third analysis encompassing all control (healthy+benign) vs PDAC samples. For all three analyses, the model was fitted for the corresponding training set. Internal data splitting with bootstrap cross-validation was used to avoid any overfitting and to generate robust estimates of sensitivities and specificities. As a single split into training and test sets may not provide robust estimates, a Monte Carlo cross-validation approach was employed. This involved averaging the performance across 1000 splits to ensure that the achieved performance does not depend on specific patients ending up in the training and test sets. The performance characteristics of PancRISK were evaluated and compared in terms of the area under the ROC curve (AUC) and SNs at a fixed SPs as well as the SPs at fixed SNs being determined for all three analyses. The performance of the models designed in this study was assessed at clinically relevant SN and SP cutoffs set at >75%. Confidence intervals (95% CIs) for AUCs were derived based on DeLong's asymptotically exact method to evaluate the uncertainty of an AUC. ¹⁴ SN and SP and 95% CI were derived using non-parametric stratified resampling with the percentile method with 100 bootstrap replicates. The method provides stable estimates with lower bias than a split-sample procedure, as described by Harrell et al. and endorsed by Steyerberg et al. Positive and negative predictive values (PPV and NPV) for several prevalence estimates were calculated using a standard approach.

### Results

### LDT Analytical Validation

The developed PancSure test is based on semi-quantitative ELISAs that measure the levels of two biomarkers (LYVE1 and REG1B) in urine. The analytical validation of the LDT assay included the testing of following performance characteristics: precision, accuracy, linearity, sensitivity, specificity, reference range and the reportable range.

### Analytical Precision

The analytical precision of the developed ELISA assay was established as intra-assay and inter-assay coefficient of variation (CV) percentages. Six different samples: two with low-value, two with mid-value, and two with high-value for each of the three biomarkers were evaluated. Intra-assay precision was determined by testing all six samples in three duplicate sets in a single assay run, while inter-assay precision was determined by testing the same six samples in duplicate over three consecutive assay runs. All obtained values for each biomarker are shown in Table 3. The data are expressed as a range between the lowest and the highest CV %: for LYVE1, intra-assay CV % of 2.4-6.1, and inter-assay CV % of 2.5%-11.3 were found, while for REG1B, intra- and inter-assay CV % were 1.0-8.1 and 5.5-14.1, respectively.

TABLE 3
Analytical precision of the LDT. Intra-assay and inter-assay coefficient of variation
(CV) percentages are established using six different samples: two with low-value, two
with mid-value, and two with high-value for each of the two biomarkers. Intra-assay
precision is determined by testing all six samples in three duplicate sets in a single
assay run, while inter-assay precision is determined by testing the same six samples
in duplicate over three consecutive assay runs. The last row presents the mean values,
standard deviation (SD) and CV across of all runs for each biomarker. In summary (black
boxes) the data is expressed as a range between the lowest and the highest CV %: for
LYVE1, intra-assay CV % of 2.4-6.1, and inter-assay CV % of 2.5%-11.3 are found, while
for REG1B, intra-and inter-assay CV % are 1.0-8.1 and 5.5-14.1, respectively.

| LYVE1 (ng/mL) - Intra-Assay - Replicate Set 1 | | | | LYVE1 (ng/mL) - Intra-Assay - Replicate Set 1 | | |
|---|---|---|---|---|---|---|
| | Duplicate | Duplicate | Mean | Duplicate | Duplicate | Mean |
| Sample | (A) | (B) | [(A)(B)] | (A) | (B) | [(A)(B)] |
| PDACC001 | 5.14 | 5.25 | 5.2 | 3.93 | 3.61 | 3.8 |
| PDACC002 | 28.50 | 26.79 | 2.6 | 26.38 | 26.04 | 26.2 |
| PDACC009 | 63.10 | 60..55 | 61.8 | 55.75 | 57.15 | 56.5 |
| PDACC084 | 7.11 | 6.36 | 6.7 | 5.56 | 4.98 | 5.3 |
| PDACC147 | 17.56 | 17.13 | 17.3 | 15.49 | 13.92 | 14.7 |
| PDACC153 | 11.36 | 11.45 | 11.4 | 8.66 | 9.04 | 8.8 |
| LYVE1 (ng/mL) - Intra-Assay - Replicate Set 2 | | | | LYVE1 (ng/mL) - Intra-Assay - Replicate Set 2 | | |
| | Duplicate | Duplicate | Mean | Duplicate | Duplicate | Mean |
| Sample | (A) | (B) | [(A)(B)] | (A) | (B) | [(A)(B)] |
| PDACC001 | 4.48 | 4.73 | 4.6 | 3.80 | 3.43 | 3.6 |
| PDACC002 | 25.61 | 25.92 | 25.8 | 27.84 | 27.69 | 27.8 |
| PDACC009 | 59.70 | 60.80 | 60.3 | 52.95 | 49.97 | 51.5 |
| PDACC084 | 7.31 | 6.79 | 7.0 | 5.15 | 4.75 | 5.0 |
| PDACC147 | 16.29 | 16.43 | 16.4 | 13.10 | 11.85 | 12.5 |
| PDACC153 | 9.71 | 10.96 | 10.3 | 8.08 | 8.71 | 8.4 |
| LYVE1 (ng/mL) - Intra-Assay - Replicate Set 3 | | | | LYVE1 (ng/mL) - Intra-Assay - Replicate Set 3 | | |
| | Duplicate | Duplicate | Mean | Duplicate | Duplicate | Mean |
| Sample | (A) | (B) | [(A)(B)] | (A) | (B) | [(A)(B)] |
| PDACC001 | 5.04 | 4.94 | 5.0 | 3.76 | 3.45 | 3.6 |
| PDACC002 | 26.79 | 27.04 | 26.9 | 23.59 | 22.54 | 23.1 |
| PDACC009 | 62.90 | 67.15 | 65.0 | 49.93 | 49.02 | 49.5 |
| PDACC084 | 6.88 | 6.72 | 6.8 | 6.07 | 5.32 | 5.7 |
| PDACC147 | 16.32 | 16.23 | 16.3 | 15.59 | 15.59 | 15.6 |
| PDACC153 | 10.60 | 10.51 | 10.6 | 9.52 | 10.25 | 9.9 |

| LYVE1 Precision Intra-Assay | | | | LYVE1 Precision Inter-Assay | | |
|---|---|---|---|---|---|---|
| | Duplicate | Duplicate | Mean | Duplicate | Duplicate | Mean |
| Sample | (A) | (B) | [(A)(B)] | (A) | (B) | [(A)(B)] |
| PDACC001 | 4.9 | 0.30 | 6.1% | 3.7 | 0.09 | 2.5% |
| PDACC002 | 26.8 | 0.95 | 3.5% | 25.7 | 2.39 | 9.3% |
| PDACC009 | 62.4 | 2.43 | 3.9% | 52.5 | 3.60 | 6.9% |
| PDACC084 | 6.9 | 0.16 | 2.4% | 5.3 | 0.37 | 7.0% |
| PDACC147 | 16.7 | 0.59 | 3.6% | 14.3 | 1.61 | 11.3% |
| PDACC153 | 10.8 | 0.56 | 5.2% | 9.0 | 0.76 | 8.5% |
| Range | Min | 2.4% | | Range | Min | 2.5% |
| | Max | 6.1% | | | Max | 11.3% |

| REG1B (ng/mL) - Intra-Assay - Replicate Set 1 | | | | REG1B (ng/mL) - Intra-Assay - Replicate Set 1 | | |
|---|---|---|---|---|---|---|
| | Duplicate | Duplicate | Mean | Duplicate | Duplicate | Mean |
| Sample | (A) | (B) | [(A)(B)] | (A) | (B) | [(A)(B)] |
| PDACC009 | 84.65 | 82.90 | 83..8 | 90.85 | 72.20 | 81.5 |
| PDACC018 | 61.25 | 60.50 | 60.9 | 50.45 | 46.83 | 48.6 |
| PDACC049 | 80.60 | 78.45 | 79.5 | 73.65 | 72.45 | 73.1 |
| PDACC067 | 45.16 | 46.01 | 45.6 | 33.51 | 30.84 | 32.2 |
| PDACC177 | 81.65 | 84.15 | 82.9 | 68.80 | 65.45 | 67.1 |
| PDACC208 | 84.85 | 83.60 | 84.2 | 67.85 | 68.35 | 68.1 |
| REG1B (ng/mL) - Intra-Assay - Replicate Set 2 | | | | REG1B (ng/mL) - Intra-Assay - Replicate Set 2 | | |
| | Duplicate | Duplicate | Mean | Duplicate | Duplicate | Mean |
| Sample | (A) | (B) | [(A)(B) | (A) | (B) | [(A)(B)] |
| PDACC009 | 81.15 | 83.95 | 82.6 | 64.10 | 63.85 | 64.0 |
| PDACC018 | 46.01 | 60.30 | 53.2 | 42.95 | 41.48 | 42.2 |
| PDACC049 | 83.60 | 78.65 | 81.1 | 57.65 | 54.55 | 56.1 |
| PDACC067 | 45.16 | 44.09 | 44.6 | 27.46 | 27.97 | 27.7 |
| PDACC177 | 84.85 | 78.10 | 81.5 | 55.50 | 64.30 | 59.9 |
| PDACC208 | 84.15 | 81.15 | 82.7 | 60.55 | 61.50 | 61.0 |
| REG1B (ng/mL) - Intra-Assay - Replicate Set 3 | | | | REG1B (ng/mL) - Intra-Assay - Replicate Set 3 | | |
| | Duplicate | Duplicate | Mean | Duplicate | Duplicate | Mean |
| Sample | (A) | (B) | (A)(B)] | (A) | (B) | [(A)(B)] |
| PDACC009 | 89.40 | 91.80 | 90.6 | 72.85 | 72.50 | 72.7 |
| PDACC018 | 61.45 | 62.00 | 61.7 | 49.88 | 49.46 | 49.7 |
| PDACC049 | 81.85 | 82.70 | 82.3 | 70.85 | 72.85 | 71.9 |
| PDACC067 | 44.09 | 45.37 | 44.7 | 29.69 | 26.61 | 28.1 |
| PDACC177 | 79.15 | 78.80 | 79.0 | 71.80 | 67.20 | 69.5 |
| PDACC208 | 82.35 | 85.35 | 83.9 | 61.35 | 66.25 | 63.8 |

| REG1B Precision Intra-Assay | | | | REG1B Precision Inter-Assay | | |
|---|---|---|---|---|---|---|
| | Duplicate | Duplicate | Mean | Duplicate | Duplicate | Mean |
| Sample | (A) | (B) | [(A)(B)] | (A) | (B) | [(A)(B)] |
| PDACC009 | 85.64 | 4.34 | 5.1% | 72.73 | 8.78 | 12.1% |
| PDACC018 | 58.58 | 4.72 | 8.1% | 46.84 | 4.04 | 8.6% |
| PDACC049 | 80.98 | 1.38 | 1.7% | 67.00 | 9.46 | 14.1% |
| PDACC067 | 44.98 | 0.52 | 1.2% | 29.35 | 2.46 | 8.4% |
| PDACC177 | 81.12 | 1.99 | 2.4% | 65.51 | 5.00 | 7.6% |
| PDACC208 | 83.58 | 0.82 | 1.0% | 64.31 | 3.56 | 5.5% |
| Range | Min | 1.0% | | Range | Min | 5.5% |
| | Max | 8.1% | | | Max | 14.1% |

### Analytical Accuracy

The analytical accuracy was determined using 60 representative urine samples: 20 from healthy donors, 20 from patients with benign pancreatic diseases and 20 from patients with PDAC. A comparative analysis was performed between the results generated with RUO kits and values obtained using commercially available ELISA kits. The correlation coefficients for LYVE1 and REG1B between the two sources of kits was 0.93 and 0.92, respectively (Table 4).

Table 4. Analytical accuracy of the LDT. The table shows a comparative analysis of data obtained with RUO kits and data generated using commercially available ELISA kits for the two biomarkers. The correlation coefficients for LYVE1 and REG1B between the two sources of kits are 0.93 and 0.92, respectively.

TABLE 4
Analytical accuracy of the LDT.

| | | | LYVE1 | LYVE1 |
|---|---|---|---|---|
| | | | DxD Hub | R&D Systems |
| Sample No. | Sample ID | Group | (ng/mL) | (ng/mL) |
| 1 | 1810-UR | Healthy | 18.84 | 1.81 |
| 2 | PDACCS004 | Healthy | 4.79 | 3.77 |
| 3 | PDACCS016 | Healthy | 12.47 | 9.67 |
| 4 | PDACCS018 | Healthy | 64.65 | 37.70 |
| 5 | PDACCS042 | Healthy | 58.45 | 36.40 |
| 6 | PDACCS049 | Healthy | 18.15 | 13.86 |
| 7 | PDACCS059 | Healthy | 4.86 | 4.25 |
| 8 | PDACCS067 | Healthy | 17.27 | 15.81 |
| 9 | PDACCS084 | Healthy | 4.81 | 3.48 |
| 10 | PDACCS090 | Healthy | 56.80 | 35.04 |
| 11 | PDACCS101 | Healthy | 36.26 | 19.07 |
| 12 | PDACCS118 | Healthy | 12.89 | 7.90 |
| 13 | PDACCS129 | Healthy | 6.36 | 4.02 |
| 14 | PDACCS132 | Healthy | 1.86 | 1.56 |
| 15 | PDACCS135 | Healthy | 59.80 | 26.66 |
| 16 | PDACCS209 | Healthy | 8.27 | 7.76 |
| 17 | PDACCS210 | Healthy | 4.21 | 3.39 |
| 18 | PDACCS217 | Healthy | 18.00 | 13.64 |
| 19 | PDACCS219 | Healthy | 100.00 | 60.58 |
| 20 | PDACCS221 | Healthy | 1.56 | 1.56 |
| 21 | PDACCS001 | Benign | 3.71 | 3.99 |
| 22 | PDACCS002 | Benign | 20.84 | 4.47 |
| 23 | PDACCS005 | Benign | 20.07 | 16.16 |
| 24 | PDACCS008 | Benign | 100.00 | 100.00 |
| 25 | PDACCS009 | Benign | 53.40 | 31.60 |
| 26 | PDACCS013 | Benign | 100.00 | 72.24 |
| 27 | PDACCS014 | Benign | 86.55 | 50.71 |
| 28 | PDACCS019 | Benign | 16.88 | 12.71 |
| 29 | PDACCS020 | Benign | 56.65 | 37.15 |
| 30 | PDACCS022 | Benign | 9.91 | 8.89 |
| 31 | PDACCS025 | Benign | 20.86 | 16.29 |
| 32 | PDACCS031 | Benign | 23.02 | 16.12 |
| 33 | PDACCS035 | Benign | 41.53 | 30.74 |
| 34 | PDACCS036 | Benign | 100.00 | 77.33 |
| 35 | PDACCS037 | Benign | 94.00 | 52.64 |
| 36 | PDACCS045 | Benign | 96.40 | 59.18 |
| 37 | PDACCS051 | Benign | 8.42 | 8.95 |
| 38 | PDACCS053 | Benign | 2.88 | 2.65 |
| 39 | PDACCS061 | Benign | 2.30 | 2.32 |
| 40 | PDACCS064 | Benign | 90.75 | 13.41 |
| 41 | PDACCS113 | PDAC | 100.00 | 77.53 |
| 42 | PDACCS147 | PDAC | 16.39 | 10.60 |
| 43 | PDACCS148 | PDAC | 100.00 | 76.23 |
| 44 | PDACCS149 | PDAC | 146.10 | 100.00 |
| 45 | PDACCS150 | PDAC | 100.00 | 100.00 |
| 46 | PDACCS154 | PDAC | 83.45 | 35.34 |
| 47 | PDACCS155 | PDAC | 100.00 | 67.14 |
| 48 | PDACCS158 | PDAC | 19.60 | 11.32 |
| 49 | PDACCS159 | PDAC | 100.00 | 55.86 |
| 50 | PDACCS161 | PDAC | 100.00 | 100.00 |
| 51 | PDACCS163 | PDAC | 100.00 | 100.00 |
| 52 | PDACCS165 | PDAC | 106.10 | 84.80 |
| 53 | PDACCS168 | PDAC | 32.55 | 19.70 |
| 54 | PDACCS170 | PDAC | 100.00 | 78.62 |
| 55 | PDACCS172 | PDAC | 92.75 | 52.58 |
| 56 | PDACCS184 | PDAC | 144.70 | 63.96 |
| 57 | PDACCS197 | PDAC | 100.00 | 73.44 |
| 58 | PDACCS201 | PDAC | 79.15 | 42.73 |
| 59 | PDACCS208 | PDAC | 16.54 | 12.21 |
| 60 | PDACCS211 | PDAC | 97.90 | 57.15 |

Correlation (r) Overall: 0.91

| | | | REG1B | REG1B |
|---|---|---|---|---|
| | | | DxD Hub | Sino Biological |
| Sample No. | Sample ID | Group | (ng/mL) | (ng/mL) |
| 1 | 1810-UR | Healthy | 18.46 | 4.70 |
| 2 | PDACCS004 | Healthy | 8.60 | 6.51 |
| 3 | PDACCS016 | Healthy | 7.81 | 5.08 |
| 4 | PDACCS018 | Healthy | 58.30 | 28.89 |
| 5 | PDACCS042 | Healthy | 123.35 | 56.52 |
| 6 | PDACCS049 | Healthy | 91.80 | 39.03 |
| 7 | PDACCS059 | Healthy | 7.81 | 3.13 |
| 8 | PDACCS067 | Healthy | 39.39 | 24.17 |
| 9 | PDACCS084 | Healthy | 7.81 | 3.69 |
| 10 | PDACCS090 | Healthy | 115.45 | 42.67 |
| 11 | PDACCS101 | Healthy | 39.56 | 20.23 |
| 12 | PDACCS118 | Healthy | 20.99 | 14.70 |
| 13 | PDACCS129 | Healthy | 47.08 | 24.45 |
| 14 | PDACCS132 | Healthy | 7.81 | 3.13 |
| 15 | PDACCS135 | Healthy | 53.15 | 21.84 |
| 16 | PDACCS209 | Healthy | 7.81 | 6.76 |
| 17 | PDACCS210 | Healthy | 7.81 | 6.76 |
| 18 | PDACCS217 | Healthy | 7.81 | 6.98 |
| 19 | PDACCS219 | Healthy | 18.58 | 11.95 |
| 20 | PDACCS221 | Healthy | 7.81 | 3.13 |
| 21 | PDACCS001 | Benign | 7.81 | 5.93 |
| 22 | PDACCS002 | Benign | 7.81 | 3.13 |
| 23 | PDACCS005 | Benign | 13.30 | 9.05 |
| 24 | PDACCS008 | Benign | 500.00 | 62.31 |
| 25 | PDACCS009 | Benign | 105.60 | 47.13 |
| 26 | PDACCS013 | Benign | 82.45 | 41.74 |
| 27 | PDACCS014 | Benign | 500.00 | 200.00 |
| 28 | PDACCS019 | Benign | 7.81 | 3.80 |
| 29 | PDACCS020 | Benign | 103.80 | 46.84 |
| 30 | PDACCS022 | Benign | 7.81 | 4.21 |
| 31 | PDACCS025 | Benign | 24.91 | 16.86 |
| 32 | PDACCS031 | Benign | 10.86 | 6.87 |
| 33 | PDACCS035 | Benign | 7.81 | 4.99 |
| 34 | PDACCS036 | Benign | 96.10 | 48.11 |
| 35 | PDACCS037 | Benign | 202.25 | 92.57 |
| 36 | PDACCS045 | Benign | 102.50 | 40.72 |
| 37 | PDACCS051 | Benign | 7.81 | 3.59 |
| 38 | PDACCS053 | Benign | 7.81 | 3.13 |
| 39 | PDACCS061 | Benign | 7.81 | 3.13 |
| 40 | PDACCS064 | Benign | 500.00 | 200.00 |
| 41 | PDACCS113 | PDAC | 500.00 | 200.00 |
| 42 | PDACCS147 | PDAC | 7.81 | 3.13 |
| 43 | PDACCS148 | PDAC | 500.00 | 200.00 |
| 44 | PDACCS149 | PDAC | 500.00 | 200.00 |
| 45 | PDACCS150 | PDAC | 500.00 | 200.00 |
| 46 | PDACCS154 | PDAC | 468.80 | 178.13 |
| 47 | PDACCS155 | PDAC | 500.00 | 200.00 |
| 48 | PDACCS158 | PDAC | 10.06 | 3.40 |
| 49 | PDACCS159 | PDAC | 24.29 | 6.91 |
| 50 | PDACCS161 | PDAC | 500.00 | 200.00 |
| 51 | PDACCS163 | PDAC | 500.00 | 200.00 |
| 52 | PDACCS165 | PDAC | 45.74 | 18.74 |
| 53 | PDACCS168 | PDAC | 500.00 | 200.00 |
| 54 | PDACCS170 | PDAC | 500.00 | 156.80 |
| 55 | PDACCS172 | PDAC | 204.35 | 73.20 |
| 56 | PDACCS184 | PDAC | 500.00 | 200.00 |
| 57 | PDACCS197 | PDAC | 50.75 | 7.98 |
| 58 | PDACCS201 | PDAC | 124.65 | 25.43 |
| 59 | PDACCS208 | PDAC | 78.20 | 43.44 |
| 60 | PDACCS211 | PDAC | 500.00 | 3.13 |

Correlation (r) Overall: 0.92

### Analytical Linearity

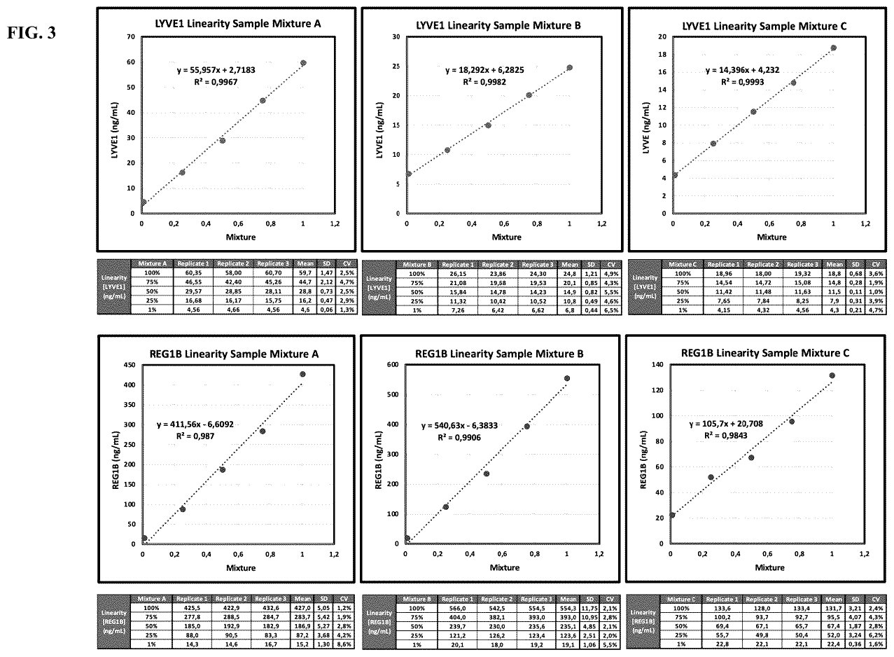

The analytical linearity of each biomarker was determined using six urine samples combined in three mixture pools (A), (B) and (C). Each pool was prepared utilizing two samples, with one sample having a low biomarker value and the other one a high biomarker value (FIG. 3). Each pool was established with a highest-concentration sample, mixed with a lowest-concentration sample in five different proportions [100% (highest concentration), 75%, 50% 25% and 1% (lowest concentration)] according to Westgard et al. 19 The respective linearities for mixture pools [A/B/C] are: LYVE1 [R²±0.997/0.998/0.999] and REG1B [R²±0.987/0.99I/O.984].

### Analytical Sensitivity

For each biomarker assay standard curves were established with nine standard points. These were prepared by 2-fold serial dilution of recombinant LYVE1 and REG1B proteins (Table 5). Using three representative lots (RUO 5, 6, 7) in nine separate assay runs, the quantitative values of each standard's concentration were calculated and interpolated using a four-parameter 4906-6716-7920.1 Page 38 of 45 065472-000986USC1 logistic (4PL) curve fit established for the standard curves and backfitting each standard's sample concentrations.

TABLE 5
Analytical sensitivity of the LDT. The analytical sensitivity was
established for the standard curves of each biomarker assay with
nine standard points prepared by 2-fold serial dilution of recombinant
LYVE1 and REG1B proteins. The quantitative values of each standard’s
concentration are calculated and interpolated using a four-parameter
logistic (4PL) curve fit. Under the acceptance criteria of a recovery
percentage limit of 100% ± 20%, and an assay imprecision of
CV < 20.0%, the analytical accuracy for LYVE1 and REG1B standard
curves ranges from lower limit of quantitation (LLOQ) to upper limit
of quantitation (ULOQ) as follows: for LYVE1, 1.56 ng/ml-100 ng/ml
and for REG1B, 7.81 ng/ml-500 ng/ml. Standards that do not meet the
criteria are bolded. “<Curve” means out of range.
LYVE1 - Standard Curve Recovery

| | Target | | | | | *Target |
|---|---|---|---|---|---|---|
| | LYVE1 | | | | Recovery | LYVE1 |
| Standard | (pg/mL) | Mean | SD | CV | Percentage | (ng/mL) |
| S1 | 2000 | 1999.9 | 1.9 | 0.1% | 100.0% | 100.00 |
| S2 | 1000 | 1003.4 | 4.1 | 0.4% | 100.3% | 50.00 |
| S3 | 500 | 495.6 | 7.3 | 1.5% | 99.1% | 25.00 |
| S4 | 250 | 249.1 | 5.7 | 2.3% | 99.6% | 12.50 |
| S5 | 125 | 129.6 | 6.5 | 5.0% | 103.7% | 6.25 |
| S6 | 62.5 | 67.5 | 4.7 | 7.0% | 108.0% | 3.13 |
| S7 | 31.25 | 33.2 | 1.8 | 5.4% | 106.2% | 1.56 |
| S8 | 15.63 | 11.5 | 4.0 | 34.7% | 73.6% | 0.78 |
| S9 | 7.813 | <Curve | NA | NA | NA | 0.4 |

REG1B - Standard Curve Recovery

| | Target | | | | | **Target |
|---|---|---|---|---|---|---|
| | REG1B | | | | Recovery | REG1B |
| Standard | (pg/mL) | Mean | SD | CV | Percentage | (ng/mL) |
| S1 | 1000 | 999.8 | 0.7 | 0.1% | 100.0% | 500.00 |
| S2 | 500 | 500.8 | 2.7 | 0.5% | 100.2% | 250.00 |
| S3 | 250 | 249.2 | 4.6 | 1.9% | 99.7% | 125.00 |
| S4 | 125 | 124.2 | 3.1 | 2.5% | 99.4% | 62.50 |
| S5 | 62.5 | 63.9 | 3.2 | 5.0% | 102.3% | 31.25 |
| S6 | 31.3 | 32.7 | 4.9 | 15.1% | 104.7% | 15.63 |
| S7 | 15.63 | 16.1 | 2.2 | 13.5% | 103.3% | 7.81 |
| S8 | 7.81 | 7.8 | 2.6 | 33.4% | 100.2% | 3.91 |
| S9 | 3.906 | <Curve | NA | NA | NA | 1.95 |

*Corrected for sample dilution factor and unit conversion, rounded to two decimal places: e.g. 2000 pg/mL = 2.00 ng/ml; 2.00 ng/ml × 50 (LYVE1 sample dilution factor) = 100 ng/ml.
**Corrected for sample dilution factor and unit conversion, rounded to two decimal places: e.g. 1000 pg/mL = 1 ng/ml; 1 ng/ml × 500 (REG1B sample dilution factor) = 500 ng/ml.

Applying the acceptance criteria combining the recovery percentage limits of 100%±20%, and assay imprecision of CV<20.0%, the analytical accuracy for LYVE1 and REG1B standard curves (ranging from lower limit of quantitation (LLOQ) to upper limit of quantitation (ULOQ)) were 1.56 ng/ml-100 ng/ml for LYVE1 and 7.81 ng/ml-500 ng/ml for REG1B.

### Analytical Specificity (Interfering Substances)

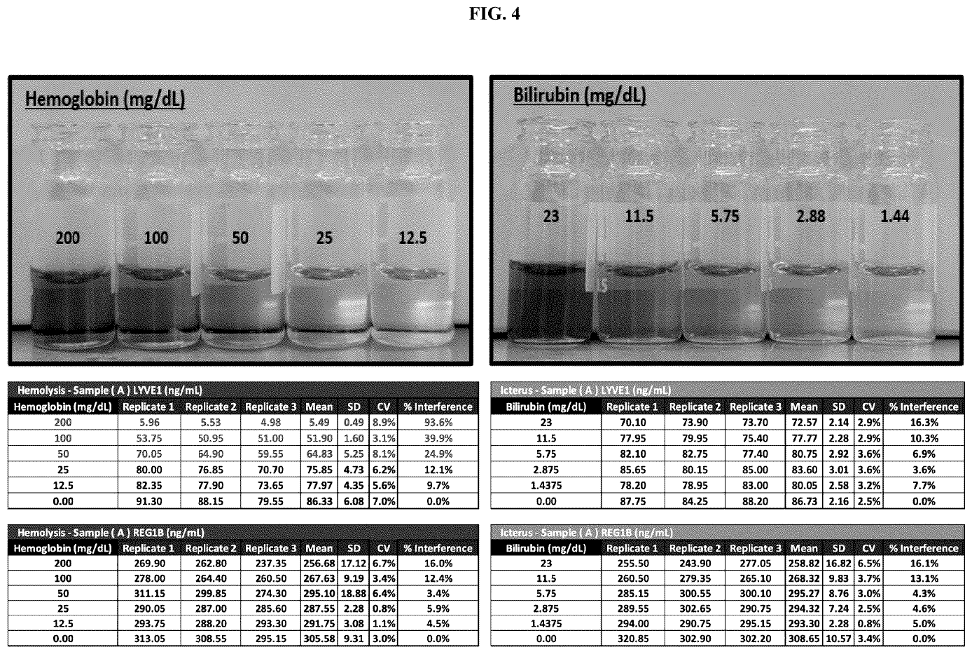

The analytical specificity of the LYVE1 and REG1B assays by interfering substances was examined, specifically hemoglobin due to hemolysis and bilirubin due to icterus that can appear in the urine of PDAC patients. For each biomarker three urine samples were utilized, representing low, mid, and high biomarker values. The sample and the interfering substances (2-fold serial dilution over five points) were tested as 50%/50% mixture proportions, using stock solutions for hemolysis (hemoglobin) and icterus (bilirubin) from the Liquichek Serum Indices (Biorad, cat. no. 12012693, 12012694). The results are shown in the FIG. 4. It is evident that no significant interference (>20%) was observed for LYVE1 and REG1B measurements with bilirubin levels up to 23 mg/dl, and for REG1B with hemoglobin levels up to 200 mg/dl. However, at very high hemoglobin levels of 50 mg/dl and above, interference was observed for LYVE1 measurements: 40% at 100 mg/dl, and 93% at 200 mg/dl. The testing and interpretation of data obtained from such highly hemolytic samples would thus require special caution.

### Biomarker Ranges in Urine

The reference ranges for LYVE1 and REG1B were established by testing 117 urine samples from healthy donors. Outliers were defined as data points that fell below Q1-1.5 IQR or above Q3+1.5 IQR. Outliers were excluded from reference range calculation. The reference ranges for LYVE1 and REG1B were established at the 2.5th percentile distribution (lower limit) and 97.5th percentile distribution (upper limit) and were 1.56 ng/ml-79.63 ng/ml, and 7.81 ng/ml-97.93 ng/ml, respectively. Samples from healthy donors were sorted by the original sample ID and named arbitrarily as H-001 through H-117.

The reportable ranges of LYVE1 and REG1B were fixed by the LLOQ and ULOQ determined in analytical sensitivity study and are 1.56 ng/ml-100 ng/ml for LYVE1, and 7.81 ng/ml-500 ng/ml for REG1B.

The biological ranges for the two biomarkers were set by testing 206 PDAC samples. Outliers were defined by data points that fell below Q1-1.5 IQR or above Q3+1.5 IQR. The data showed non-Gaussian distribution. Thus, the biological ranges were created at the 2.5th percentile distribution (lower limit) and 97.5th percentile distribution (upper limit) as follows: LYVE1: 3.59-100.00 ng/ml, and REG1B: 7.81-500.00 ng/ml.

### LDT Clinical Validation

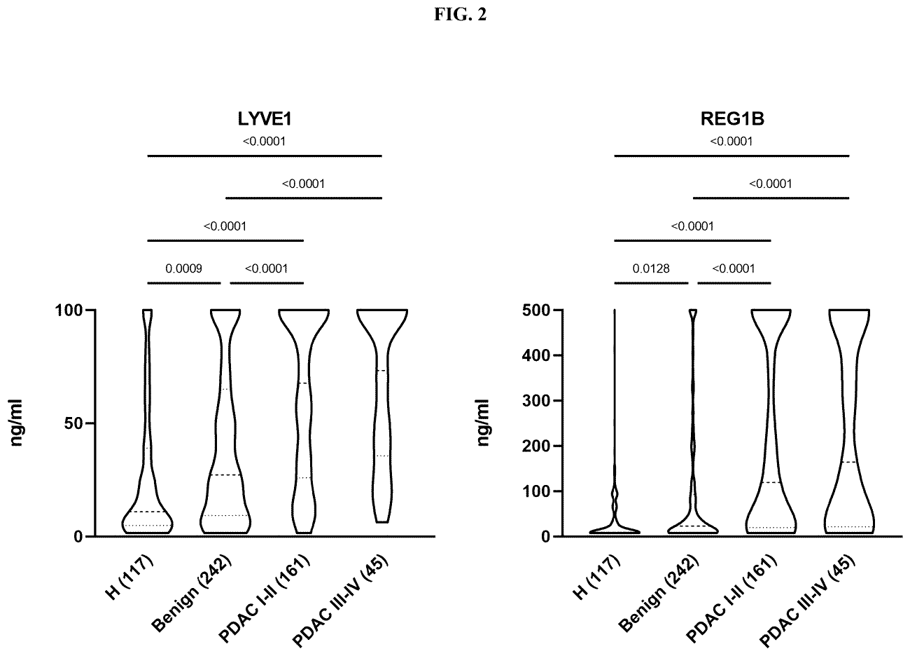

The LDT clinical validation was performed in a cohort of 565 patients. A significantly higher concentration of both biomarkers in PDAC specimens when compared with both healthy control and benign samples was confirmed (Kruskal-Wallis test, p<0.0001) as shown by violin plots in FIG. 2. While the biomarker values were higher in earlier PDAC stages, this difference did not reach statistical significance.

Three supervised statistical methods: logistic regression, support vector machine (SVM) and random forest were tested and compared with the input of the concentrations of LYVE1, REG1B, and CA19.9, plus age for best performance. The area under the receiver operating characteristic (ROC) curve (AUC) values for the three statistical methods across all three comparisons—healthy (H) vs PDAC, benign (Be) vs PDAC, and (H+Be) vs PDAC—are summarized in Table 6. As the methods performed similarly, logistic regression was utilized as before.

TABLE 6
Comparative results of different supervised statistical methods.
The results are shown for the following three comparisons:
Healthy (H) vs Pancreatic Ductal Adenocarcinoma (PDAC), Benign
(Be) vs PDAC and combined (H + Be) vs PDAC.

| | H vs PDAC | Be vs PDAC | H + Be vs PDAC |
|---|---|---|---|
| Logistic Regression | 0.93 | 0.86 | 0.89 |
| | (0.91-0.97) | (0.82-0.92) | (0.85-0.93) |
| SVM | 0.93 | 0.85 | 0.87 |
| | (0.91-0.97) | (0.81-0.91) | (0.83-0.93) |
| Random Forest | 0.93 | 0.86 | 0.89 |
| | (0.77-0.9) | (0.64-0.77) | (0.7-0.82) |

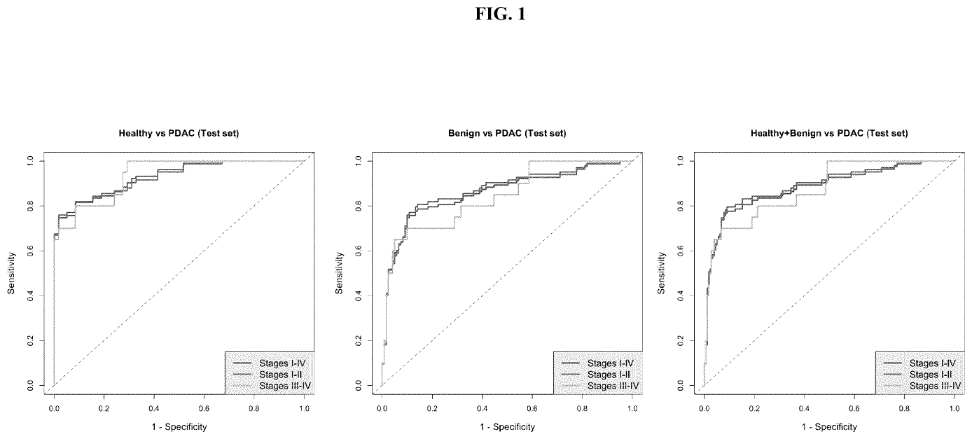

A summary of the performance of the adapted PancRISK in the test set (averaged across 100 random 50:50 splits) is presented as ROC curves (FIG. 1). To demonstrate that the accuracy of the test is not due to age itself, the following calculations were performed: a) relative contribution of the variables to the overall AUC by summarizing the AUCs for each variable independently; b) age stratified AUC results of the two biomarkers+/−CA 19.9 when fitted with a logistic model without age and c) correlation of the biomarkers with age (Table 7).

Table 7. Contribution of age to the predictive model. A) shows univariate analysis and AUC values in the validation set for each biomarker as well as reported full model, whereas B) displays the comparison of the full model with the reduced model that does not contain age; C) presents the correlation of each of the biomarkers with age in a full dataset of healthy and PDAC patients (without splitting into training and test sets).

| Model | AUC (95% CI) |
|---|---|
| LYVE1 | 0.78 (0.7-0.85) |
| REG1B | 0.78 (0.72-0.85) |
| CA19-9 | 0.89 (0.83-0.94) |
| Age | 0.77 (0.7-0.85) |
| LYVE1 + REG1B + CA19- | 0.93 (0.91-0.97) |
| 9 + Age | |

| Model | AUC (95% CI) |
|---|---|
| LYVE1 + REG1B + CA19-9 | 0.91 (0.87-0.95) |
| LYVE1 + REG1B + CA19- | 0.93 (0.91-0.97) |
| 9 + Age | |

| Covariate | Correlation with Age |
|---|---|
| LYVE1 | 0.27 (p < 0.001) |
| REG1B | 0.26 (p < 0.001) |
| CA19-9 | 0.16 (p = 0.05) |

Table 8 shows the performance of the PancRISK for three comparisons: H vs PDAC, Be vs PDAC, and non-malignant (H+Be) vs PDAC, for a range of fixed SNs and SPs. In H vs PDAC model, SNs are listed for SPs up to 99% (as opposed to the rest of the following tables) as high specificity is desired to avoid a high-false positive rate.

Positive and negative predictive values (PPV and NPV) for varied prevalences at fixed SN/SP across each comparison are displayed in Table 9.

Healthy vs PDAC: Sensitivity based on fixed Specificity

| Specificity | All Stages | Early Stages | Late Stages |
|---|---|---|---|
| 0.75 | 0.92 (0.86-0.96) | 0.91 (0.85-0.96) | 0.96 (0.88-1) |
| 0.80 | 0.89 (0.82-0.93) | 0.89 (0.82-0.93) | 0.93 (0.82-1) |
| 0.85 | 0.86 (0.79-0.91) | 0.86 (0.78-0.91) | 0.87 (0.75-1) |
| 0.90 | 0.82 (0.74-0.88) | 0.82 (0.73-0.89) | 0.81 (0.66-0.92) |
| 0.95 | 0.76 (0.66-0.84) | 0.77 (0.66-0.86) | 0.73 (0.55-0.84) |
| 0.96 | 0.76 (0.66-0.84) | 0.77 (0.66-0.86) | 0.73 (0.55-0.84) |
| 0.97 | 0.74 (0.63-0.83) | 0.75 (0.64-0.84) | 0.71 (0.54-0.84) |
| 0.98 | 0.74 (0.63-0.83) | 0.75 (0.64-0.84) | 0.71 (0.54-0.84) |
| 0.99 | 0.71 (0.61-0.83) | 0.72 (0.61-0.83) | 0.68 (0.5-0.83) |

Healthy vs PDAC: Specificity based on fixed Sensitivity

| Sensitivity | All Stages | Early Stages | Late Stages |
|---|---|---|---|
| 0.75 | 0.96 (0.89-1) | 0.97 (0.9-1) | 0.95 (0.86-1) |
| 0.80 | 0.93 (0.84-1) | 0.93 (0.84-1) | 0.92 (0.82-1) |
| 0.85 | 0.87 (0.78-0.95) | 0.87 (0.76-0.97) | 0.88 (0.79-0.96) |
| 0.90 | 0.8 (0.69-0.88) | 0.78 (0.66-0.87) | 0.83 (0.72-0.92) |
| 0.95 | 0.65 (0.52-0.78) | 0.6 (0.43-0.77) | 0.78 (0.64-0.9) |

Benign vs PDAC: Sensitivity based on fixed Specificity

| Specificity | All Stages | Early Stages | Late Stages |
|---|---|---|---|
| 0.75 | 0.83 (0.78-0.89) | 0.85 (0.8-0.9) | 0.77 (0.65-0.9) |
| 0.80 | 0.81 (0.75-0.87) | 0.82 (0.75-0.88) | 0.76 (0.65-0.86) |
| 0.85 | 0.78 (0.72-0.84) | 0.79 (0.72-0.86) | 0.75 (0.63-0.86) |
| 0.90 | 0.73 (0.65-0.82) | 0.73 (0.65-0.83) | 0.73 (0.6-0.86) |
| 0.95 | 0.59 (0.47-0.72) | 0.58 (0.45-0.71) | 0.64 (0.48-0.83) |

Benign vs PDAC: Specificity based on fixed Sensitivity

| Sensitivity | All Stages | Early Stages | Late Stages |
|---|---|---|---|
| 0.75 | 0.87 (0.8-0.93) | 0.88 (0.81-0.93) | 0.84 (0.64-0.96) |
| 0.80 | 0.81 (0.69-0.91) | 0.83 (0.71-0.91) | 0.75 (0.56-0.94) |
| 0.85 | 0.69 (0.59-0.84) | 0.72 (0.58-0.85) | 0.65 (0.47-0.87) |
| 0.90 | 0.55 (0.37-0.67) | 0.54 (0.29-0.72) | 0.56 (0.34-0.72) |
| 0.95 | 0.35 (0.2-0.54) | 0.3 (0.16-0.51) | 0.47 (0.26-0.64) |

Healthy + Benign vs PDAC: Sensitivity based on fixed Specificity

| Specificity | All Stages | Early Stages | Late Stages |
|---|---|---|---|
| 0.75 | 0.85 (0.8-0.9) | 0.86 (0.8-0.91) | 0.8 (0.68-0.91) |
| 0.80 | 0.83 (0.78-0.87) | 0.84 (0.78-0.9) | 0.78 (0.67-0.88) |
| 0.85 | 0.81 (0.75-0.86) | 0.82 (0.75-0.89) | 0.76 (0.62-0.88) |
| 0.90 | 0.77 (0.71-0.83) | 0.78 (0.7-0.84) | 0.74 (0.6-0.87) |
| 0.95 | 0.66 (0.57-0.75) | 0.65 (0.55-0.76) | 0.66 (0.52-0.79) |

Healthy + Benign vs PDAC: Specificity based on fixed Sensitivity

| Sensitivity | All Stages | Early Stages | Late Stages |
|---|---|---|---|
| 0.75 | 0.91 (0.84-0.95) | 0.91 (0.85-0.96) | 0.86 (0.69-0.96) |
| 0.80 | 0.85 (0.75-0.93) | 0.87 (0.77-0.94) | 0.78 (0.63-0.95) |
| 0.85 | 0.75 (0.64-0.87) | 0.78 (0.63-0.9) | 0.71 (0.56-0.94) |
| 0.90 | 0.62 (0.45-0.75) | 0.63 (0.41-0.82) | 0.62 (0.43-0.78) |
| 0.95 | 0.44 (0.28-0.61) | 0.39 (0.21-0.63) | 0.55 (0.34-0.74) |

TABLE 9
Positive and negative predictive values for the
three diagnostic models for differing prevalences.
PPV and NPV: Calculated for different Diagnostic Models

| Model | Prevalence | Specificity | Sensitivity | PPV | NPV |
|---|---|---|---|---|---|
| H vs PDAC | 0.005 | 0.98 | 0.74 | 0.16 | 0.999 |
| | 0.010 | | | 0.27 | 0.997 |
| | 0.020 | | | 0.43 | 0.995 |
| Be vs PDAC | 0.005 | 0.85 | 0.78 | 0.03 | 0.999 |
| | 0.010 | | | 0.05 | 0.997 |
| | 0.020 | | | 0.10 | 0.995 |
| H + Be vs PDAC | 0.005 | 0.85 | 0.81 | 0.03 | 0.999 |
| | 0.010 | | | 0.05 | 0.998 |
| | 0.020 | | | 0.10 | 0.995 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of calculating a risk score for a subject, comprising:
obtaining urine concentrations of lymphatic vessel endothelial hyaluronan receptor 1 (LYVE1) protein, and regenerating family member 1 beta (REG1B) protein;
obtaining the subject's age;
obtaining blood concentration of CA19.9 carbohydrate; and
calculating the risk score using the following Formula Ib2:
$$\frac{1}{( 1 + {e}^{( - ( β 0 ) + ( β 1 * L ) + ( β 2 * R ) + ( β 3 * C ) + ( β 4 * A ) ) )} )} \qquad [ Formula Ib2 ]$$
wherein
L is log_{c}(concentration of LYVE1±0.01),
R is log_{c}(concentration REG1B+0.01),
C is log_{c}(concentration CA19.9-+0.01), and
A is age of subject, and
wherein the calculation for β is: β=b*SD_{IV}*R/SD_{LogitY} and wherein
β is the standardized weight,
b is the unstandardized weight,
SD_{IV} is the standard deviation of the independent variable,
R is the Pearson correlation coefficient, and
SD_{LogitY} is the standard deviation of the log odds predicted probability, and
wherein
β0 is −6.895079±0.75,
β1 is 0.007487015±0.005,
β2 is 0.3566643±0.5,
β3 is 0.7100223±0.5, and β4 is 0.0399479±0.05.

2. A The method of calculating a risk score for a subject of claim 1 wherein the method comprises:
obtaining urine concentrations of lymphatic vessel endothelial hyaluronan receptor 1 (LYVE1) protein, and regenerating family member 1 beta (REG1B) protein;
obtaining the subject's age;
obtaining blood concentration of CA19.9 carbohydrate; and
calculating the risk score using the following Formula Ia:
$$\frac{1}{( 1 + {e}^{( - ( - 6.895079 - ( 0.007487015 * L ) + ( 0.3566643 * R ) + ( 0.7100223 * C ) + ( 0.0399479 * A ) ) )} )}$$
wherein
L is log_{c}(concentration of LYVE1±0.01),
R is log_{c}(concentration REG1B+0.01),
C is log_{c}(concentration CA19.9 protein+0.01), and
A is age of subject.

3. The method of claim 2, further comprising:
comparing the risk score to a threshold score; and
stratifying a subject as having pancreatic cancer if the risk score is equal to or higher than the threshold score, or stratifying a subject as not having pancreatic cancer if the risk score is lower than the threshold score.

4. The method of claim 2, wherein the threshold score is 0.412.

5. The method of claim 2, wherein the method does not include obtaining urine concentrations of TFF1.

6. A method of testing a subject in need thereof, comprising:
detecting concentration of LYVE1 protein in a urine sample and comparing the LYVE1 protein concentration to a reference range for a healthy subject, or comparing the LYVE1 protein concentration to a biological range for a cancer subject;
detecting concentration of REG1B protein in a urine sample and comparing the REG1B protein concentration to a reference range for a healthy subject, or comparing the REG1B protein concentration to a biological range for a cancer subject;
detecting concentration of CA19.9 carbohydrate in a blood sample; and
obtaining the subject's age.

7-12. (canceled)

13. The method of claim 1, wherein obtaining urine concentrations of LYVE1 and REG1B comprises:
contacting a urine sample, obtained from the subject, to an assay, the assay comprising:
one enzyme-linked immunosorbent assay (ELISA) for measuring the concentrations of LYVE1 and REG1B, or
two ELISAs, a first ELISA for measuring the concentration of LYVE1 and a second ELISA for measuring the concentration of REG1B.

14. The method of claim 13, wherein
the one ELISA comprises an antibody that specifically binds to LYVE1 and an antibody that specifically binds to REG1B, or
the two ELISAs comprise the first ELISA comprising an antibody that specifically binds to LYVE1 and the second ELISA comprising an antibody that specifically binds to REG1B.

15. The method of claim 13, wherein
the one ELISA comprises a primary capture antibody that specifically binds to LYVE1 and a primary capture antibody that specifically binds to REG1B, or
the two ELISAs comprise the first ELISA comprising a primary capture antibody that specifically binds to LYVE1 and the second ELISA comprising a primary capture antibody that specifically binds to REG1B.

16. The method of claim 15, wherein
the one ELISA further comprises a secondary detection antibody pair that specifically binds to LYVE1 including an additional antibody conjugated to a reporter molecule and a secondary detection antibody pre-conjugated with a reporter molecule that specifically binds to REG1B, or
the two ELISAs comprise the first ELISA further comprising a secondary detection antibody pair that specifically binds to LYVE1 including an additional antibody conjugated to a reporter molecule and the second ELISA further comprising a secondary detection antibody pre-conjugated with a reporter molecule that specifically binds to REG1B.

17. The method of claim 1, wherein obtaining urine concentrations of LYVE1 and REG1B comprises using an antibody that specifically binds to LYVE1 and an antibody that specifically binds to REG1B.

18. The method according to of claim 1, wherein the urine sample or blood sample is obtained from a human subject having or suspected of having pancreatic ductal adenocarcinoma (PDAC), or a human subject having one or more genetic predispositions to PDAC development, a human subject having one or more symptoms of pancreatic cancer.

19. The method of claim 18, wherein the pancreatic ductal adenocarcinoma is stage I, stage II, stage III or stage IV pancreatic ductal adenocarcinoma.

20. The method of claim 1, wherein obtaining blood concentration of CA19.9 carbohydrate comprises
contacting a blood sample, obtained from the subject, to an enzyme-linked immunosorbent assay (ELISA) or a chemiluminescence assay for measuring the concentration of CA19.9 carbohydrate.

21. The method of claim 1, wherein obtaining blood concentration of CA19.9 protein comprises using an immunoassay.

22. The method of claim 1, wherein the method does not include obtaining urine concentrations of TFF1.

23. The method of claim 1, comprising stratifying a subject as having pancreatic cancer if the risk score is equal to or higher than the threshold score and performing invasive diagnostic tests on the subject to further confirm the presence of PDAC.

24. The method of claim 1, comprising stratifying a subject as having pancreatic cancer if the risk score is equal to or higher than the threshold score and further comprising administering PDAC therapy to the subject.

25. A kit for testing for pancreatic ductal adenocarcinoma, comprising:
one enzyme-linked immunosorbent assay (ELISA) for measuring the concentration of Lymphatic vessel endothelial hyaluronan receptor 1 (LYVE1) and Regenerating islet-derived 1B (REG1B), or
two ELISAs, a first ELISA for measuring concentration of LYVE1, and a second ELISA for measuring concentration of REG1B.

26-30. (canceled)

31. A combination, comprising the kit of claim 25, and a urine sample.

32. (canceled)

33. A method for confirming pancreatic ductal adenocarcinoma (PDAC) in a subject, and optionally treating the subject, comprising:
obtaining a risk score calculated by the method of claim 1, or calculating a risk score by performing the method of claim 1;
determining the risk score to be equal or higher than a threshold score;
performing one or more imaging test or one or more biopsies on the subject to further confirm the presence of PDAC.

34. The method of claim 33, further comprising administering PDAC therapy to the subject.

35-37. (canceled)

38. A method for further confirming pancreatic ductal adenocarcinoma (PDAC) in a subject, and optionally treating the subject, comprising:
performing one or more imaging test or one or more biopsies on a subject who has been determined to have a risk score equal to or higher than a threshold score; and optionally administering a PDAC therapy to a subject.

39. The method of claim 38, wherein the risk score is calculated by:
obtaining urine concentrations of lymphatic vessel endothelial hyaluronan receptor 1 (LYVE1) protein, and regenerating family member 1 beta (REG1B) protein;
obtaining the subject's age;
obtaining blood concentration of CA19.9 carbohydrate; and
calculating the risk score using the following Formula Ib2:
$$\frac{1}{( 1 + {e}^{( - ( β 0 ) + ( β 1 * L ) + ( β 2 * R ) + ( β 3 * C ) + ( β 4 * A ) ) )} )} \qquad [ Formula Ib2 ]$$
wherein
L is log_{c}(concentration of LYVE1±0.01),
R is log_{c}(concentration REG1B+0.01),
C is log_{c}(concentration CA19.9-+0.01), and
A is age of subject, and
wherein the calculation for β is: β=b*SD_{IV}*R/SD_{LogitY} and wherein
β is the standardized weight,
b is the unstandardized weight,
SD_{IV} is the standard deviation of the independent variable,
R is the Pearson correlation coefficient, and
SD_{LogitY} is the standard deviation of the log odds predicted probability, and
wherein
β0 is −6.895079±0.75,
β1 is 0.007487015±0.005,
β2 is 0.3566643±0.5,
β3 is 0.7100223±0.5, and
β4 is 0.0399479±0.05
